(19) United States
(12) Patent Application Publication
(10) Pub. No.: US 2009/0105285 A1
(43) Pub. Date: Apr. 23, 2009

(54) PHARMACEUTICAL COMBINATIONS COMPRISING A MTOR INHIBITOR AND A RAF KINASE INHIBITOR

(75) Inventor: Heidi Lane, Biel-Benken (CH)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 12/299,819

(22) PCT Filed: May 9, 2007
(86) PCT No.: PCT/EP07/04112

§ 371 Date: Nov. 6, 2008

(30) Foreign Application Priority Data
May 11, 2006 (GB) 0609378.5

(51) Int. Cl. A61K 31/517; A61K 31/436

(52) U.S. Cl. 514/266.21; 514/294

(57) ABSTRACT

A pharmaceutical combination comprising an mTOR inhibitor and a Raf kinase inhibitor and its use.

The present invention relates to organic compounds, e.g. a combination of pharmaceutically active compounds.

It was found that a combination of an mTOR inhibitor with a Raf kinase inhibitor show surprising activities, e.g. synergistic activity, in cancer treatment.

In one aspect the present invention provides a combination of an mTOR inhibitor and a Raf kinase inhibitor.

A combination of an mTOR inhibitor and a Raf kinase inhibitor is herein also designated as “A combination of (according to) the present invention.”

An mTOR inhibitor is a compound which targets intracellular mTOR (“mammalian Target of rapamycin”). mTOR is a family member of phosphatidylinositol 3-kinase(P13-kinase) related kinase. The compound rapamycin and other mTOR inhibitors inhibit the mTOR pathway via a complex with its intracellular receptor FKBP12 (FK506-binding protein 12). mTOR modulates translation of specific mRNAs via the regulation of the phosphorylation state of several different translation proteins, mainly 4E-PB1, P70S6K (p70S6 kinase 1) and eEF2.

An mTOR inhibitor of (according to) the present invention e.g. includes rapamycin, which is a known macrolide antibiotic produced by Streptomyces hygroscopicus, and rapamycin derivatives, e.g. rapamycin substituted in position 40 and/or 16 and/or 32, for example a compound of formula

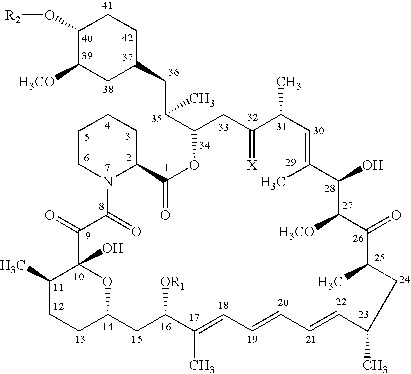

wherein
R₁ is CH₃ or C₃₋₆alkynyl,

### R₂ is H, —CH₂—CH₂—OH, or —CH₂—CH₂O—CH₂—CH₃.

3-hydroxy-2-(hydroxymethyl)-2-methyl-propanoyl or tetrazolyl, e.g. tetrazol-1-yl, and
X is ═O, (H, H) or (H, OH), provided that R₂ is other than H when X is ═O and R₁ is CH₃. When R₂ in a compound of formula I is —CH₂—CH₂—OH, a compound of formula I includes a physiologically hydrolysable ether thereof, for instance —CH₂—CH₂—O—(C₁₋₈)alkyl.

Representative examples of compounds of formula I include e.g. 40-O-(2-hydroxy)ethyl-rapamycin (also known as everolimus), 32-deoxorapamycin, 16-O-substituted rapamycins such as 16-pent-2-ynyloxy-32-deoxorapamycin, 16-pent-2-ynyloxy-32 (S or R)-dihydro-rapamycin, 16-pent-2-ynyloxy-32 (S or R)-dihydro-40-0-(2-hydroxyethyl)-rapamycin, 40-[3-hydroxy-2-(hydroxy-methyl)-2-methylpropanoate]-rapamycin (also known as CCI779), 40-epi-(tetrazolyl)-rapamycin (also known as ABT578), or 40-O-ethoxyethyl-rapamycin (also known as biolimus 9).

mTOR inhibitors also include the so-called rapalogs, e.g. as disclosed in WO9802441, WO0114387 and WO0364383, such as AP23573, e.g. 40-O-(dimethylphosphinyl)-rapamycin, compounds as disclosed disclosed in WO2005047295 in Example 1, also designated as biolimus A9 and compounds disclosed under the name TAFA-93.

Other mTOR inhibitors are e.g. disclosed in WO2004101583, WO9205179, WO9402136, WO9402385, WO9613273.

Preferably mTOR inhibitors include rapamycin, a compound of formula I, e.g. and including a rapalog, TAFA-93, more preferably rapamycin, a compound of formula I or a rapalog.

A preferred compound is e.g. 40-O-(2-hydroxyethyl)-rapamycin disclosed in Example 8 in WO9409010.

Another preferred compound is 32-deoxorapamycin or 16-pent-2-ynyloxy-32 (S)-dihydro-rapamycin as disclosed in WO9641807, e.g. or a compound as disclosed in WO9516691.

Preferred mTOR inhibitors include

rapamycin, and/or
40-O-(2-hydroxyethyl)-rapamycin, and/or
32-deoxorapamycin, and/or
16-pent-2-ynyloxy-32-deoxorapamycin, and/or
16-pent-2-ynyloxy-32 (S or R)-dihydro-rapamycin, and/or
16-pent-2-ynyloxy-32 (S or R)-dihydro-40-0-(2-hydroxyethyl)-rapamycin, and/or
40-[3-hydroxy-2-(hydroxy-methyl)-2-methylpropanoate]-rapamycin (also known as CCI779) and/or
40-epi-(tetrazolyl)-rapamycin (also known as ABT578), and/or
AP23573, and/or
biolimus A9, e.g. and/or
compounds disclosed under the name TAFA-93,
such as
40-O-(2-hydroxyethyl)-rapamycin, and/or
32-deoxorapamycin, and/or
CCI779, and/or
ABT578, and/or

### AP23573.

mTOR inhibitors, e.g. rapamycin or rapamycin derivatives, may be administered as appropriate, e.g. in the form of pharmaceutical composition, such as as disclosed, e.g. in dosages which are known for rapamycin or rapamycin derivatives, e.g. everolimus may be administered, e.g. orally, in dosages from 0.1 mg up to 15 mg, such as 0.1 mg to 10 mg. e.g. 0.1 mg, 0.25 mg, 0.5 mg, 0.75 mg, 1 mg, 2.5 mg, 5 mg, or 10 mg, e.g. in the form of (dispersible) tablets; e.g. a weekly dosage may include up to 70 mg, e.g. 30 mg to 70 mg, such as 30 mg to 50 mg; depending on the disease being treated. Other rapamycin derivatives may be administered in similar dosage ranges, e.g. in dosage ranges such as disclosed.

The Raf kinase family is known to have three members designated cRaf, also known as Raf-1, bRaf and aRaf. It has been reported that bRaf kinase is commonly activated by one of several somatic point mutations in human cancer, including 59% of the melanoma cell lines tested, see e.g. Davies et al., Nature, Vol. 417, pp. 949-954 (2002). Raf kinase inhibitors as used herein are meant to include efficient inhibitors of RAF kinase, particularly cRAF kinase inhibitors and wild and mutated bRAF kinase inhibitors, e.g. including inhibitors of the V599E mutant bRAF kinase.

Raf kinase inhibitors, e.g. low molecular compounds, are known. Raf kinase inhibitors including such as described herein, are herein also designated as “Raf kinase inhibitors of (according to) the present invention”.

Preferred Raf kinase inhibitors according to the present invention e.g. include the compounds GW5074, BAY 43-9006, CHIR-265, and compounds as defined in U.S. Pat. No. 6,987,119 (which compounds are described to be particularly B-Raf kinase inhibitors), WO98022103, WO99032436, WO2006084015, WO2006125101, WO2007027855, WO2005004864, WO2005028444, WO03082272, WO2005032548, and WO2007030377, more preferably compounds as defined in WO2005028444, WO03082272, WO2005032548, and WO2007030377.

It is specifically referred herein to the compounds as disclosed in any form, e.g. whether disclosed in a general formula, or whether disclosed as single compounds, e.g. whether disclosed in free base form or whether disclosed in the form of salts, solvates, polymorphs, esters, N-oxides, prodrugs, isomer mixtures or pure isomers, or tautomers, of the patent filings cited herein; and each group of compounds disclosed or each single compound disclosed therein may be a preferred Raf kinase inhibitor according to the present invention. It is further specifically referred to production processes and pharmaceutical compositions and mode of administration as disclosed for any compound group or single compound in any of the patent filing cited herein. The content of the patent filings cited herein is introduced by reference.

In another aspect the present invention provides a combination of an mTOR inhibitor with a compound as disclosed in WO2005028444 which is a compound of formula

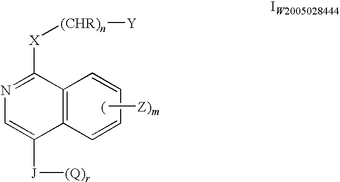

wherein
n is from 0-2;
r is from 0 to 2,
m is from 0-4;
J is unsubstituted or substituted once or twice by Q, wherein J is aryl, heteroaryl, cycloalkyl or heterocycloalkyl, wherein aryl is an aromatic radical having from 6-14 carbon atoms, such as phenyl, naphthyl, fluorenyl and phenanthrenyl; heteroaryl is an aromatic radical having from 4-14, especially from 5-7 ring atoms, of which 1, 2 or 3 atoms are chosen independently from N; S and 0, such as furyl, pyranyl, pyridyl, 1,2-, 1,3-and 1,4-pyrimidinyl, pyrazinyl, triazinyl, triazolyl, oxazolyl, quinazolyl, imidazolyl, pyrrolyl, isoxazolyl isothiazolyl, indolyl, isoindolinyl, quinolyl, isoquinolyl, purinyl, cinnolinyl, naphthyridinyl, phthalazinyl, isobenzofuranyl, chromenyl, purinyl, thianthrenyl, xanthenyl, acridinyl, carbazolyl and phenazinyl; cycloalkyl is a saturated cyclic radical having from 3-8, preferably from 5-6 ring atoms, such as cyclopropyl, cyclopentyl and cyclohexyl; heterocycloalkyl is a saturated cyclic radical having from 3-8, preferably from 5-6 ring atoms, of which 1, 2 or 3 atoms are chosen independently from N, S and 0, such as piperidinyl, piperazinyl, imidazolidinyl, pyrrolidinyl and pyrazolidinyl;
Q is a substituent on 1 or 2 carbon atoms selected from the group consisting of halogen, unsubstituted or substituted lower alkyl, —OR₂, —SR₂, —N(R)R, —NRS(O)₂N(R)R, —NRS(O)₂R, —S(O)R₂, —S(O)₂R₂, —OCOR₂, —C(O)R₂, —CO₂R₂, —NR—COR₂, —CON(R₂)R₂, —S(O)₂N(R₂)R₂, cyano, tri-methylsilanyl, unsubstituted or substituted aryl, unsubstituted or substituted heteroaryl, such as substituted or unsubstituted imidazolyl, and substituted or unsubstituted pyridinyl, unsubstituted or substituted cycloalkyl, unsubstituted or substituted heterocycloalkyl, such as substituted or unsubstituted piperidinyl, substituted or unsubstituted piperazolyl, substituted or unsubstituted tetrahydropyranyl, and substituted or unsubstituted azetidinyl _{C1-4}alkyl-aryl, —C₁₋₄alkyl-heteroaryl, —C₁₋₄alkyl-heterocyclyl, amino, mono-or disubstituted amino, heteroaryl-aryl;
R is H. lower alkyl or lower alkoxy-alkyl;
R₂ is unsubstituted or substituted alkyl, unsubstituted or substituted cycloalkyl, unsubstituted or substituted phenyl, —C₁₋₄alkyl-aryl, —C₁₋₄alkyl-heteroaryl or —C₁₋₄alkyl-heterocycloalkyl;
X is a bond, Y, —N(R)—, oxa, thio, sulfone, sulfoxide, sulfonamide, amide, or ureylene, preferably —NH—, —NHC(O)—, —NHC(O)NH—;
Y is H. lower alkyl, substituted or unsubstituted aryl, substituted or unsubstituted heteroaryl, substituted or unsubstituted cycloalkyl or substituted or unsubstituted heterocycloalkyl; and
Z is amino, mono-or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkanesulfonyl or alkylphenylsulfonyl, and where, if more than one radical Z is present (m≧2), the substituents Z are identical or different; or
an N-oxide of the mentioned compound, wherein one or more N atoms carry an oxygen atom; or a pharmaceutically acceptable salt thereof;
e.g. a compound of formula

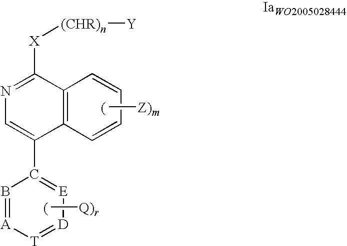

wherein r is from 0-2,
n is from 0-2;
m is from 0-4;
A, B. D, E and T are each CH or CQ, or
A, B. D and E are each CH or CQ and T is N, or
B. D, E. and T are each CH or CQ and A is N, or
A, B. T and E are each CH or CQ and D is N, or
A, B. D, and T are each CH or CQ and E is N, or
A, B and D are each CH or CQ and E and T are N, or
B. E, and T are each CH or CQ and A and D are each N, or
A, D and T are each CH or CQ and B and E are each N, or
A and D are each CH or CQ and B, E and T are each N;
Q is a substituent on 1 or 2 carbon atoms selected from the group consisting of halogen, unsubstituted or substituted lower alkyl, —OR₂, —SR₂, —N(R)R, —NRS(O)₂N(R)R, —NRS(O)₂R, —S(O)R₂, —S(O)₂R₂, —OCOR₂, —C(O)R₂, —CO₂R₂, —NR—COR₂, —CON(R₂)R₂, —S(O)₂N(R₂)R₂, cyano, tri-methylsilanyl, unsubstituted or substituted aryl, unsubstituted or substituted heteroaryl, such as substituted or unsubstituted imidazolyl, and substituted or unsubstituted pyridinyl, unsubstituted or substituted cycloalkyl, unsubstituted or substituted heterocycloalkyl, such as substituted or unsubstituted piperidinyl, substituted or unsubstituted piperazolyl, substituted or unsubstituted tetrahydropyranyl, and substituted or unsubstituted azetidinyl, —C₁₋₄alkyl-aryl, —C₁₋₄alkyl-heteroaryl, —C₁₋₄alkyl-heterocyclyl, amino, mono-or disubstituted amino, heteroaryl-aryl;
R is H, lower alkyl or lower alkoxy-alkyl;
R₂ is unsubstituted or substituted alkyl, unsubstituted or substituted cycloalkyl, unsubstituted or substituted phenyl, —C₁₋₄alkyl-aryl, —C₁₋₄alkyl-heteroaryl or —C₁₋₄alkyl-heterocycloalkyl;
X is a bond, Y, —N(R)—, oxa, thio, sulfone, sulfoxide, sulfonamide, amide, or ureylene, preferably —NH—, —NHC(O)—, —NHC(O)NH—;
Y is H, lower alkyl, substituted or unsubstituted aryl, substituted or unsubstituted heteroaryl, substituted or unsubstituted cycloalkyl or substituted or unsubstituted heterocycloalkyl; and
Z is amino, mono-or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfnyl, phenylsulfonyl, phenyl-lower alkanesulfonyl or alkylphenylsulfonyl, and where, if more than one radical Z is present (m≧2), the substituents Z are identical or different; or
an N-oxide or a pharmaceutically acceptable salt thereof;
e.g. a compound of formula Ia_{WO2005028444}, wherein
r is from 0-2;
n is 0 or 1;
m is 0 or 1;
A, B. D and E are each CH or CQ and T is N, or
A, B. T and E are each CH or CQ and D is N, or
A, B and D are each CH or CQ and E and T are each N;
Q is a substituent on one or two carbon atoms selected from the group consisting of halogen, unsubstituted or substituted lower alkyl, —OR₂, —SR₂, —NR₂, —NRS(O)₂N(R)₂, —NRS(O)₂R, S(O)R₂, —S(O)₂R₂, —COR₂, —C(O)R₂, —CO₂R₂, —NR—COR₂, —CON(R₂)₂, —S(O)₂N(R₂)₂, cyano, tri-methylsilanyl, unsubstituted or substituted aryl, unsubstituted or substituted heteroaryl, unsubstituted or substituted cycloalkyl, unsubstituted or substituted heterocycloalkyl, —C₁₋₄alkyl-aryl, —C₁₋₄alkyl-heteroaryl, —C₁₋₄alkyl-heterocyclyl, amino, mono-or disubstituted amino;
R is H or lower alkyl,
R₂ is unsubstituted or substituted alkyl, unsubstituted or substituted cycloalkyl, phenyl, —C₁₋₄alkyl-aryl, —C₁₋₄alkyl-heteroaryl, or —C₁₋₄alkyl-heterocycloalkyl;
X is —NR—, oxa or thia;
Y is phenyl that is unsubstituted or substituted by one or two identical or different substituents selected from the group consisting of amino; lower alkanoylamino, halogen, lower alkyl, halo-lower alkyl, hydroxy; lower alkoxy, phenyl-lower alkoxy, and cyano, or alternatively or additionally to the preceding group of substituents, lower alkenyl, C₈₋₁₂alkoxy, lower alkoxycarbonyl, carbamoyl, lower alkylcarbamoyl, lower alkanoyl, halo-lower alkyloxy, lower alkoxycarbonyl, lower alkylmercapto, halo-lower alkylmercapto, hydroxy-lower alkyl, lower alkanesulfonyl, halo-lower alkanesulfonyl, phenylsulfonyl, dihydroxybora (—B(OH)₂) and lower alkylenedioxy, or
Y is pyridyl; and
Z is halogen, amino, N-lower alkylamino, hydroxy-lower alkylamino, phenyl-lower alkylamino, N,N-di-lower alkylamino, N-phenyl-lower alkyl-N-lower alkylamino, N,N-di-lower alkylphenylamino, lower alkanoylamino, or a substituent selected from the group consisting of benzoylamino and phenyl-lower alkoxycarbonylamino, wherein the phenyl radical in each case is unsubstituted or is substituted by nitro or by amino, or also by halogen, amino, N-lower alkylamino, N,N-di-lower alkylamino, hydroxy, cyano, carboxy, lower alkoxycarbonyl, lower alkanoyl or by carbamoyl; or
an N-oxide or a pharmaceutically acceptable salt thereof;
e.g. a compound of formula Ia_{WO2005028444}, wherein
r is from 0-2;
n is 0 or 1;
m is 0 or 1;
A, B. D and E are each CH or CQ and T is N, or
A, B and D are each CH or CQ and E and T are each N;
Q is bonded to A, to D or to A and D; and is selected from fluorine, chlorine or bromine, methyl, ethyl, propyl; hydroxy, methoxy, ethoxy, 2-hydroxyethoxy, 2-methoxyethoxy, (2-(1H-imidazol-1-yl)ethoxy, hydroxyiminomethyl, acetyl, formyl, methylmercapto, or amino, N-methylamino, N-ethylamino, N-n-propyl- or N-isopropylamino, 2-cyanoethylamino, 3-(methoxyphenyl)amino, 3-(4-morpholinyl)propylamino, 3-(pyridinyl)methylamino, 2-(2-pyridinyl)ethylamino, 4-(1H-imidazol-1-yl)butylamino, 4-(trifluommethox)phenyl-amino, (methylaminosulfonyl)amino, (methylsulfonyl)amino, (tetrahydro-2H-pyran-4-yl)amino, (tetrahydro-2H-pyran-4-yl)methylamino, (tetrahydro-3-furanyl)amino, (2-(1H-imidazol-1-yl)ethyl)amino, 2-hydroxyethylamino, (2-methoxyethyl)methylamino, 2-(2-hydroxyethoxy)ethylamino, spirans, 1-azetidinyl, 3-ethoxycarbonyl-1-azetidinyl, 3-carboxy-1-azetidinyl, tetrahydro-2H-1,3-oxazinyl, dihydro-1,2,5-oxathiazine-5(6H)-yl, tetrahydro-1(2H)-pyrimidinyl), 3-(acetyl)-tetrahydro-1(2H)-pyrimidinyl, piperazinyl, 4-(2-hydroxyethyl)-1-piperazinyl, 4-(ethoxycarbonyl)-1-piperazinyl, 4-acetyl-1-piperazinyl, piperidinyl, 4-(trifluormethyl)-1-piperidinyl, 4-(difluoromethyl)-1-piperidinyl, 4-(phenylmethyl)-1-piperidinyl, 4-phenoxy-1-piperidinyl, 4-cyano-1-piperidinyl, 4-methoxy-1-piperidinyl, 4-ethoxycarbonyl-1-piperidinyl, 4-hydroxy-1-piperidinyl, 4-carboxy-1-piperidinyl, 4-(aminocarbonyl)-1-piperidinyl, 4-methylthio-1-piperidinyl, 4-methylsulfonyl-1-piperidinyl, (tetrahydro-2H-pyran-4-yl)oxy, 4-morpholinyl, 3,5-dimethylmorpholinyl or 2-phenyl-4-morpholinyl;
R is H or methyl;
X is —NR—, oxa or thia;
Y is phenyl that is unsubstituted or substituted by one or two identical or different substituents selected from amino; acetylamino; fluorine, chlorine or bromine; tert-butyl, methyl, ethyl or propyl; trifluoromethyl; hydroxy; methoxy, ethoxy; benzyloxy; cyano, or (alternatively or additionally to the preceding group of substituents) ethenyl, C₆₋₂alkoxy, tert-butoxycarbonyl, carbamoyl, N-methyl-carbamoyl or N-tert-butyl-carbamoyl, acetyl, phenyloxy, trifluoromethoxy, 1,1,2,2-tetRafluoroethyloxy, ethoxycarbonyl, methylmercapto, trifluoromethylmercapto, hydroxymethyl, methanesulfonyl, trifluoromethanesulfonyl, phenylsulfonyl, dihydroxybora (—B(OH)₂), 2-methyl-pyrimidin-4-yl, oxazol-5-yl, 2-methyl-1,3-dioxolan-2-yl, 1H-pyrazol-3-yl, 1-methyl-pyrazol-3-yl, methylenedioxy bonded to two adjacent carbon atoms or
Y is pyridyl, 2-, 3- or 4-aminophenyl, 2-, 3- or 4-acetylaminophenyl, 2-, 3- or 4-fluorophenyl, 2-, 3- or 4-chlorophenyl, 2-, 3- or 4-bromophenyl, 2,3-, 2,4-, 2,5- or 3,4-dichlorophenyl, chloro-fluoro-phenyl, 4-chloro-2-fluoroanilino, 2-, 3- or 4-methylphenyl, 2-, 3- or 4-ethylphenyl, 2-, 3 or 4-propylphenyl, methyl-fluoro-phenyl, 2-, 3- or 4-trifluoromethylphenyl, 2-, 3- or 4-hydroxyphenyl, 2-, 3- or 4-methoxyphenyl, 2-, 3- or 4-ethoxyphenyl, methoxy-chloro-phenyl, 2-, 3- or 4-benzyloxyphenyl, 2-, 3- or 4-cyanophenyl, 2-, 3- or 4-methylphenyl, 4-chloro-5-trifluoromethylphenyl, 3-bromo-5-trifluoromethylphenyl, 3,5-dimethylphenyl, 4-methyl-3-iodophenyl, 3,4-bis(trifluoromethyl)phenyl, 3-bromo-4-ethyl-phenyl or 3-chlorobenzylphenyl; and
Z is halogen, amino, N-lower alkylamino, hydroxy-lower alkylamino, phenyl-lower alkylamino, N,N-di-lower alkylamino, N-phenyl-lower alkyl-N-lower alkylamino; N,N-di-lower alkylphenylamino, lower alkanoylamino or a substituent selected from the group consisting of benzoylamino and phenyl-lower alkoxycarbonylamino, wherein the phenyl radical in each case is unsubstituted or, is substituted by nitro or by amino, or also by halogen, amino, N-lower alkylamino, N,N-di-lower alkylamino, hydroxy, cyano, carboxy, lower alkoxycarbonyl, lower alkanoyl or by carbamoyl; or
an N-oxide, or a pharmaceutically acceptable salt thereof.
e.g. a compound of formula Ia_{WO2005028444}, wherein
r is 1; n is 0; m is 0;

### B, D, E and T are CH or CQ and A is N, or

A, B. D and E are each CH or CQ and T is N;
Q is a substituent on one or two carbon atoms selected from fluorine, chlorine, methyl, ethyl, propyl; amino, N-methylamino, N-ethylamino, N-n-propylamino, N-isopropylamino, 2-cyanoethylamino, 3-(methoxyphenyl)amino, 3-(4-morpholinyl)propylamino, 3-(pyridinyl)methylamino, 2-(2-pyridinyl)ethylamino, 4-(1H-imidazol-1-yl)butylamino, 4-(trifluoromethoxyphenyl)amino), (methylaminosulfonyl)amino, (methylsulfonyl)amino, (tetrahydro-2H-pyran-4-yl)amino, (tetrahydro-2H-pyran-4-yl)methylamino, (tetrahydro-3-furanyl)amino, (2-(1H-imidazol-1-yl)ethyl)amino, 2-hydroxyethylamino, 2-(2-hydroxyethoxy) ethylamino, tetrahydro-1-(2H)-pyrimidinyl, 3-(acetyl)tetrahydro-1 (2H)-pyrimidinyl, piperazinyl, 4-(2-hydroxyethyl)-1-piperazinyl, 4-(ethoxycarbonyl)-1-piperazinyl, 4-acetyl-1-piperazinyl, piperidinyl, 4-(trifluormethyl)-1-piperidinyl, 4-(difluoromethyl)-1-piperidinyl, 4-(phenylmethyl)-1-piperidinyl, 4-phenoxy-1-piperidinyl, 4-cyano-1-piperidinyl, 4-methoxy-1-piperidinyl, 4-ethoxycarbonyl-1-piperidinyl, 4-hydroxy-1-piperidinyl, 4-carboxy-1-piperidinyl, 4-(aminocarbonyl)-1-piperidinyl, 4-methylthio-1-piperidinyl, 4-methylsulfonyl-1-piperidinyl, 4-morpholinyl, 3,5-dimethylmorpholinyl or 2-phenyl-4-morpholinyl;
R is H or methyl,

### X is —NH—; and

Y is phenyl that is unsubstituted or substituted by one or two identical or different substituents selected from fluorine, chlorine, bromine; lower alkyl, trifluoromethyl; 4-chlorophenyl, 2-, 3- or 4-methylphenyl, 4-chloro-5-trifluoromethylphenyl, 3-bromo-5-trifluoromethylphenyl, 3,5-dimethylphenyl; 4-methyl-3-iodophenyl, 3,4-bis(trifluoromethyl)phenyl or 3-bromo-4-ethyl-phenyl; or
an N-oxide or pharmaceutically acceptable salt thereof;
e.g. a compound of formula Ia_{WO2005028444}, wherein
r is 1; n is 0; m is 0;
A, B. D and E are each CH or CQ and T is N;
Q is a substituent on one carbon atom selected from amino, N-methylamino, N-ethylamino, N-n-propylamino, N-isopropylamino, 2-cyanoethylamino, 3-(methoxyphenyl)amino, 3-(4-morpholinyl)propylamino, 3-(pyridinyl)methylamino, 2-(2-pyridinyl)-ethylamino, 4-(1H-imidazol-1-yl)butylamino, 4-(trifluormethox)yphenyl-amino), (methylaminosulfonyl)amino, (methylsulfonyl)amino, (tetrahydro-2H-pyran-4-yl)amino, (tetrahydro-2H-pyran-4-yl)methylamino, (tetrahydro-3-furanyl)amino, (2-(1H-imidazol-1-ylmethyl)amino, 2-hydroxyethylamino, 2-(2-hydroxyethoxy)ethylamino, piperidinyl, 4-(trifluormethyl)-1-piperidinyl, 4-(difluoromethyl)-1-piperidinyl, 4-(phenylmethyl)-1-piperidinyl, 4-phenoxy-1-piperidinyl, 4-cyano-1-piperidinyl, 4-methoxy-1-piperidinyl, 4-ethoxycarbonyl-1-piperidinyl, 4-hydroxy-1-piperidinyl, 4-carboxy-1-piperidinyl, 4-(aminocarbonyl)-1-piperidinyl, 4-methylthio-1-piperidinyl, 4-methylsulfonyl-1-piperidinyl or morpholinyl;

### R is H;

### X is —NH—; and

Y is phenyl that is unsubstituted or substituted by chlorine, methyl, trifluoromethyl, isopropyl, tert-butyl, methoxy, 4-trifluoromethoxyphenyl; naphthyl; cyclohexyl that is unsubstituted or substituted by lower alkyl, indolyl that is unsubstituted or substituted by halogen or by lower alkyl; or
an N-oxide or pharmaceutically acceptable salt thereof;
e.g. a compound of formula Ia_{WO2005028444}, wherein
r is 1; n is 0; m is 0;
A, B. D, and E are each CH and T is N;
Q is a substituent on one carbon atom selected from morpholinyl;
R is H; X is —NH—; and Y is phenyl that is substituted in the 4-position by tert-butyl or trifluoromethyl; or
an N-oxide or pharmaceutically acceptable salt thereof;
e.g. a compound of formula Ia_{WO2005028444}, wherein
r is 1; n is 0; m is 0;
A, B and D are each CH, and E and T are each N;

### X is —NH—;

Y is phenyl that is substituted in the 4-position by tert-butyl; and
Q is a 2-hydroxyethylamino substituent on D; or
an N-oxide or pharmaceutically acceptable salt thereof;
such as a compound of formula I_{WO2005028444}, wherein
n is from 0-2;
r is from 0-2;
m is from 0-4;
J is a bicyclic heteroaromatic ring system, selected from indolyl, isoindolinyl, quinolyl, isoquinolyl, quinazolyl, purinyl, cinnolinyl, naphthyridinyl, phthalazinyl, isobenzofuranyl naphthyridinyl, phthalazinyl, chromenyl and purinyl;
Q is a substituent on either one or both rings of the bicyclic ring system, and on one or two carbon atoms on either one or both rings of the bicyclic ring system, selected from the group consisting of halogen, unsubstituted or substituted lower alkyl, —OR₂, —SR₂, —NR₂, —NRS(O)₂N(R)₂, —NRS(O)₂R, —S(O)_{R2}, —S(O)₂R₂, —OCOR₂, —C(O)R₂, —CO₂R₂, —NR—COR₂, —CON(R₂)₂, —S(O)₂N(R₂)₂, cyano, tri-methylsilanyl, unsubstituted or substituted aryl, unsubstituted or substituted heteroaryl, unsubstituted or substituted cycloalkyl, unsubstituted or substituted heterocycloalkyl, —C₁₋₄alkyl-aryl, —C₁₋₄alkyl-heteroaryl, —C₁₋₄alkyl-heterocyclyl, amino, mono-or all-substituted amino;
R is H or lower alkyl;
R₂ is unsubstituted or substituted alkyl, unsubstituted or substituted cycloalkyl, phenyl, —C₁₋₄alkyl-aryl, —C₁₁alkyl-heteroaryl or —C₁₋₄alkyl-heterocycloalkyl;
X is Y.—N(R)—, oxa, thio, sulfone, sulfoxide, sulfonamide, amide or ureylene;
Y is H. lower alkyl, substituted or unsubstituted aryl, substituted or unsubstituted heteroaryl, substituted or unsubstituted cycloalkyl or substituted or unsubstituted heterocycloalkyl; and Z is amino, mono-or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-di-substituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkanesulfonyl or alkylphenylsulfonyl, and where, if more than one radical Z is present (m≧2), the substituents Z are identical or different; or an N-oxide or a pharmaceutically acceptable salt thereof,
e.g. a compound of formula I_{WO2005028444}, wherein
n is 0; r is 0; m is 0;
J is a bicyclic heteroaromatic ring system, selected from indolyl, isoindolinyl, quinolyl, isoquinolyl, quinazolyl, purinyl, cinnolinyl, naphthyridinyl, phthalazinyl, isobenzofuranyl naphthyridinyl, phthalazinyl, chromenyl and purinyl;
R is H or lower alkyl;
X is Y.—N(R)—, oxa, thio, sulfone, sulfoxide, sulfonamide, amide or ureylene; and
Y is H. lower alkyl, substituted or unsubstituted aryl, substituted or unsubstituted heteroaryl, substituted or unsubstituted cycloalkyl or substituted or unsubstituted heterocycloalkyl; or
an N-oxide or a pharmaceutically acceptable salt thereof;
e.g. a compound of formula I_{WO2005028444}, wherein n is 0; r is 0; m is O; J is isoquinolyl; X is NH; and Y is 4-tert-butylphenyl; or
an N-oxide or a pharmaceutically acceptable salt thereof;
e.g. a compound of formula I_{WO2005028444}, wherein n is 0; r is 0; m is O; J is quinazolyl; X is NH;
and Y is 4-tert-butylphenyl;
or an N-oxide or a pharmaceutically acceptable salt thereof;
e.g. a compound of formula I_{WO2005028444}, wherein n is 0; r is O; m is O; J is isoquinolyl; X is NH; and Y is 2-tert-butyl-pyrimidin-5-yl;
or an N-oxide or a pharmaceutically acceptable salt thereof;
e.g. a compound of formula I_{WO2005028444}, which is selected from the compounds of examples 1 to 30, or from the compounds 1 to 332 in TABLE 2, or from compounds 1 to 5 in TABLE 3 as defined in WO2005028444;
e.g. or an N-oxide or a pharmaceutically acceptable salt thereof;
e.g. wherein each single compound listed may be a preferred compound;
e.g. a compound selected from the group of compounds of formula

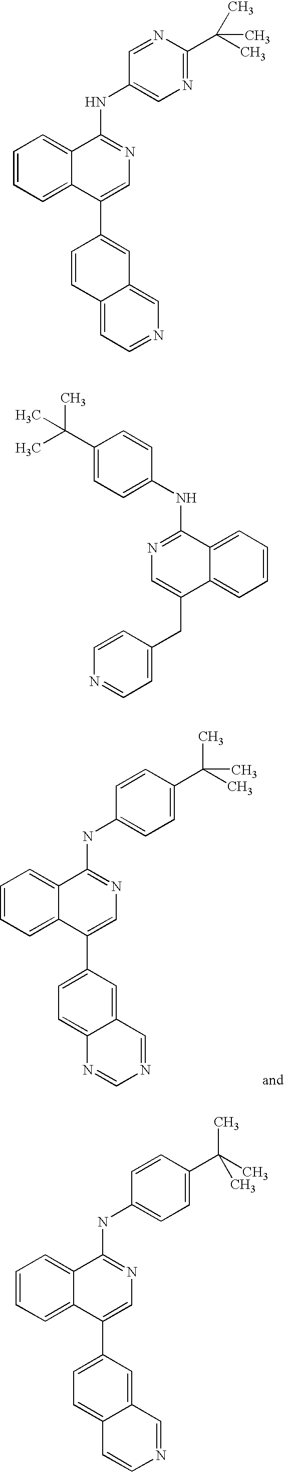

Preferred meanings of substituents of a compound of formula I_{WO2005028444} or of formula Ia_{WO2005028444}, e.g. including the meaning of the substituents n, m, r, J, Q, R, R₂, X, Y, Z, A, B, D, E and T, as indicated herein, are as defined in WO200502844 and it is referred herein to WO200502844; and the content of WO200502844 is herein introduced by reference.

The compounds of WO200502844 may be administered to a subject in need thereof as described in WO200502844. In the case of a body weight of approximately 70 kg, a daily dose of from approximately 0.1 g to approximately 5 g, preferably from approximately 0.5 g to approximately 2 g, of a compound of formula I_{WO2005028444} or of formula Ia_{WO2005028444} may be administered to a subject in need thereof, e.g. in the form of a pharmaceutical composition as defined in WO200502844.

it is referred herein to WO200502844 in any aspect; and the content of WO200502844 is herein introduced by reference.

In another aspect the present invention provides a combination of an mTOR inhibitor with a compound as disclosed in WO03082272 or WO2005032548, which is a compound of formula

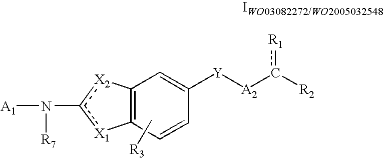

wherein X₁ and X₂ are independently of each other selected from ═N—, —NR₄—, —O— or —S—, provided that
if X₁ is —NR₄—, —O— or —S—, then X₂ is —N—, or
if X₂ is —NR₄—, —O— or —S—, then X₁ is ═N—, and both X₁ and X₂ are not ═N—;

### Y is O or S;

A₁ is substituted or unsubstituted alkyl, cycloalkyl, heterocycloalkyl, aryl, polycyclic aryl, polycyclic arylalkyl, heteroaryl, biaryl, heteroarylaryl, heteroarylheteroaryl, cycloalkylalkyl, heterocycloalkylalkyl, arylalkyl, heteroarylalkyl, diarylalkyl, or heteroarylarylalkyl;
A₂ is substituted or unsubstituted heteroaryl;

### R₁ is O or H. and

R₂ is NR₅R₆ or hydroxyl; or
R₁ and R₂ together with the carbon atom to which they are attached form a substituted or unsubstituted heterocycloalkyl or heteroaryl group; wherein, the dotted line represents a single or double bond;
R₃ is hydrogen, halogen, loweralkyl, or loweralkoxy;

R₄ is hydrogen, hydroxyl, alkylamino, dialkylamino or alkyl;

R₅ and R₆ are independently of each other selected from hydrogen, and substituted or unsubstituted alkyl, alkoxyalkyl, aminoalkyl, amidoalkyl, acyl, cycloalkyl, heterocycloalkyl, aryl, heteroaryl, alkyloxyalkylheterocyclyl, and heteroarylalkyl; or
R₅ and R₆ together with the nitrogen atom to which they are attached form substituted or unsubstituted heterocyclyl or heteroaryl; and
R₇ is hydrogen or loweralkyl,
or a pharmaceutically acceptable salt, ester or prodrug thereof;
e.g. including a compound of formula I_{WO03082272/WO2005032548}, wherein
X is NR₄, e.g. and wherein R₄ is hydrogen or methyl;
Y is O;
A₁ is selected from the group consisting of substituted or unsubstituted phenyl, pyridyl, pyrimidinyl, phenylalkyl, pyridylalkyl, pyrimidinylalkyl, heterocyclylcarbonylphenyl, heterocyclylphenyl, heterocyclylalkylphenyl, chlorophenyl, fluorophenyl, bromophenyl, iodophenyl, dihalophenyl, nitrophenyl, 4-bromophenyl, 4-chlorophenyl, alkylbenzoate, alkoxyphenyl, dialkoxyphenyl, dialkylphenyl, trialkylphenyl, thiophene, thiophene-2-carboxylate, alkylthiophenyl, trifluoromethylphenyl, acetylphenyl, sulfamoylphenyl, biphenyl, cyclohexylphenyl, phenyloxyphenyl, dialkylaminophenyl, alkylbromophenyl, alkylchlorophenyl, alkylfluorophenyl, trifluoromethylchlorophenyl, trifluoromethylbromophenyl indenyl, 2,3-dihydroindenyl, tetralinyl, trifluorophenyl, (trifluoromethyl)thiophenyl, alkoxybiphenyl, morpholinyl, N-piperazinyl, N-morpholinylalkyl, piperazinylalkyl, cyclohexylalkyl, indolyl, 2,3-dihydroindolyl, 1-acetyl-2,3-dihydroindolyl, cycloheptyl, bicyclo[2.2.1]hept-2-yl, hydroxyphenyl, hydroxyalkylphenyl, pyrrolidinyl, pyrrolidin-1-yl, pyrrolidin-1-ylalkyl, 4-amino(imino)methylphenyl, isoxazolyl, indazolyl, adamantyl, bicyclohexyl, quinuclidinyl, imidazolyl, benzimidazolyl, imidazolylphenyl, phenylimidazolyl, pthalamido, napthyl, benzophenone, anilinyl, anisoyl, quinolinyl, quinolinonyl, phenylsulfonyl, phenylalkylsulfonyl, 9H-flouren-1-yl,
piperidin-1-yl, piperidin-1-ylalkyl, cyclopropyl, cyclopropylalkyl, pyrimidin-5-ylphenyl, quinolidinylphenyl, furanyl, furanylphenyl, N-methylpiperidin-4-yl, pyrrolidin-4-ylpyridinyl, 4-diazepan-1-yl, hydroxypyrrolidin-1-yl, dialkylaminopyrrolidin-1-yl, 1,4′-bipiperidin-1′-yl, and (1,4′-bipiperidin-1′-ylcarbonyl)phenyl;
   A₂ is substituted or unsubstituted pyridyl,
   R₁ is O and the dotted line represents a single or double bond;
   R₂ is NR₅R₆, R₅ is hydrogen and R₆ is selected from hydrogen, and substituted or unsubstituted alkyl, alkoxyalkyl, aminoalkyl, amidoalkyl, acyl, cycloalkyl, heterocycloalkyl, aryl, heteroaryl, alkyloxyalkylheterocyclo, and heteroarylalkyl;
   R₁ and R₂ together with the carbon atom to which they are attached form a substituted or unsubstituted heterocycloalkyl or heteroaryl group;
   R₃ is loweralkoxy; such as methoxy.
   R₄ is loweralkyl, such as methyl;
   R₁ is O, R₂ is NR₅R₆, R₅ is H. and R₆ is methyl;
      e.g. including a compound of formula I_{WO03082272/WO2005032548}, which is a compound of formula

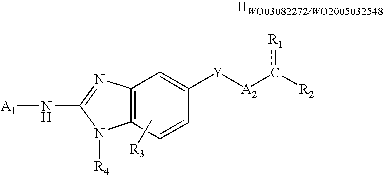

wherein Y is O or S;
A₁ is substituted or unsubstituted cycloalkyl, heterocycloalkyl, aryl, polycyclic aryl, polycyclic arylalkyl, heteroaryl, biaryl, heteroarylaryl, heteroarylheteroaryl, cycloalkylalkyl, heterocycloalkylalkyl, arylalkyl, heteroarylalkyl, biarylalkyl, heteroarylarylalkyl;
A₂ is substituted or unsubstituted heteroaryl;

### R₁ is O and R₂ is NR₅R₆; or

R₁ and R₂ together with the carbon atom to which they are attached form a substituted or unsubstituted heterocycloalkyl or heteroaryl group; wherein, the dotted line represents a single or double bond;
R₃ is hydrogen, halogen, loweralkyl, or loweralkoxy;
R₄ is hydrogen or loweralkyl,
R₅ and R₆ independently of each other are selected from hydrogen, and substituted or unsubstituted alkyl, alkoxyalkyl, aminoalkyl, amidoalkyl, acyl, cycloalkyl, heterocycloalkyl, aryl, heteroaryl, alkyloxyalkylheterocyclo, and heteroarylalkyl; or
R₅ and R₆ together with the nitrogen atom to which they are attached form substituted or unsubstituted heterocyclo or heteroaryl; or
a pharmaceutically acceptable salt, ester, or prodrug thereof;
e.g. including a compound of formula II_{WO03082272/WO2005032548}, wherein
R₄ is hydrogen,
R₄ is methyl,
Y is O,
A₁ is selected from the group consisting of substituted or unsubstituted phenyl, pyridyl, pyrimidinyl, phenylalkyl, pyridylalkyl, pyrimidinylalkyl, heterocyclylcarbonylphenyl, heterocyclylphenyl, heterocyclylalkylphenyl, chlorophenyl, fluorophenyl, bromophenyl, iodophenyl, dihalophenyl, nitrophenyl, 4-bromophenyl, 4-chlorophenyl, alkylbenzoate, alkoxyphenyl, dialkoxyphenyl, dialkylphenyl, trialkylphenyl, thiophene, thiophene-2-carboxylate, alkylthiophenyl, trifluoromethylphenyl, acetylphenyl, sulfamoylphenyl, biphenyl, cyclohexylphenyl, phenyloxyphenyl, dialkylaminophenyl, alkylbromophenyl, alkylchlorophenyl, alkylfluorophenyl, trifluoromethylchlorophenyl, trifluoromethylbromophenyl indenyl, 2,3-dihydroindenyl, tetralinyl, trifluorophenyl, (trifluoromethyl)thiophenyl, alkoxybiphenyl, morpholinyl, N-piperazinyl, N-morpholinylalkyl, piperazinylalkyl, cyclohexylalkyl, indolyl, 2,3-dihydroindolyl, 1 acetyl-2,3-dihydroindolyl, cycloheptyl, bicyclo[2.2.1] hept-2-yl, hydroxyphenyl, hydroxyalkylphenyl, pyrrolidinyl, pyrrolidin-1-yl, pyrrolidin-1-ylalkyl, 4-amino(imino)methylphenyl, isoxazolyl, indazolyl, adamantyl, bicyclohexyl, quinuclidinyl, imidazolyl, benzimidazolyl, imidazolylphenyl, phenylimidazolyl, pthalamido, napthyl, benzophenone, anilinyl, anisoyl, quinolinyl, quinolinonyl phenylsulfonyl, phenylalkylsulfonyl, 9H-flouren-1-yl, piperidin-1-yl, piperidin-1-ylalkyl, cyclopropyl, cyclopropylalkyl, pyrimidin-5-ylphenyl, quinolidinylphenyl, furanyl, furanylphenyl, N-methylpiperidin-4-yl, pyrrolidin-4-ylpyridinyl, 4-diazepan-1-yl,-245 hydroxypyrrolidin-1-yl, dialkylaminopyrrolidin-1-yl, 1,4′-bipiperidin-1′-yl, and (1,4′-bipiperidin-1′-ylcarbonyl)phenyl;
A₂ is substituted or unsubstituted pyridyl;
R₁ is O and the dotted line represents a single or double bond;
R₂ is NR₅R₆, R₅ is hydrogen and Rr is selected from hydrogen, and substituted or unsubstituted alkyl, alkoxyalkyl, aminoalkyl, amidoalkyl, acyl, cycloalkyl, heterocycloalkyl, aryl, heteroaryl, alkyloxyalkylheterocyclo, and heteroarylalkyl;
R₁ and R₂ together with the carbon atom to which they are attached form a substituted or unsubstituted heterocycloalkyl or heteroaryl group;
R₁ is O, R₂ is NR₅R₆, R₅ is H. and R₆ is methyl;
R₃ is loweralkoxy, e.g. methoxy;
R₄ is loweralkyl, e.g. methyl;
   e.g. including a compound of formula I_{WO03082272/WO2005032548}, which is a compound of formula

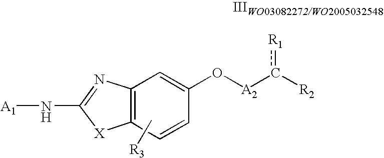

wherein

### X is NR₄, O or S.

A₁ is substituted or unsubstituted cycloalkyl, heterocycloalkyl, aryl, polycyclic aryl, polycyclic arylalkyl, heteroaryl, biaryl, heteroarylaryl, heteroarylheteroaryl, cycloalkylalkyl, heterocycloalkylalkyl, arylalkyl, heteroarylalkyl, biarylalkyl, heteroarylarylalkyl;
A₂ is substituted or unsubstituted heteroaryl;

### R₁ is O and R₂ is NR₅, R₆; or

R₁ and R₂ together with the carbon atom to which they are attached form a substituted or unsubstituted heterocycloalkyl or heteroaryl group, wherein, the dotted line represents a single or double bond;
R₃ is hydrogen, halogen, loweralkyl, or loweralkoxy;
R₄ is hydrogen or loweralkyl;
R₅ and R₆ independently of each other are selected from hydrogen and substituted or unsubstituted alkyl, alkoxyalkyl, aminoalkyl, amidoalkyl, acyl, cycloalkyl, heterocycloalkyl, aryl, heteroaryl, alkyloxyalkylheterocyclo, and heteroarylalkyl; or
R₅ and R₆ together with the nitrogen atom to which they are attached form substituted or unsubstituted heterocyclyl or heteroaryl; or
a pharmaceutically acceptable salt, ester or prodrug thereof;
e.g. including a compound of formula III_{WO03082272/WO2005032548}, wherein
X is NR₄;
R₄ is hydrogen;
R₄ is methyl;
A₁ is selected from the group consisting of substituted or unsubstituted phenyl, pyridyl, pyrimidinyl, phenylalkyl, pyridylalkyl, pyrimidinylalkyl, heterocyclylcarbonylphenyl, heterocyclylphenyl, heterocyclylalkylphenyl, chlorophenyl, fluorophenyl, bromophenyl, iodophenyl, dihalophenyl, nitrophenyl, 4-bromophenyl, 4-chlorophenyl, alkylbenzoate, alkoxyphenyl, dialkoxyphenyl, dialkylphenyl, trialkylphenyl, thiophene, thiophene-2-carboxylate, alkylthiophenyl, trifluoromethylphenyl, acetylphenyl, sulfamoylphenyl, biphenyl, cyclohexylphenyl, phenyloxyphenyl, dialkylaminophenyl, alkylbromophenyl, alkylchlorophenyl, alkylfluorophenyl, trifluoromethylchlorophenyl, trifluoromethylbromophenyl indenyl, 2,3-dihydroindenyl, tetralinyl, trifluorophenyl, (trifluoromethyl)thiophenyl, alkoxybiphenyl, morpholinyl, N-piperazinyl, N-morpholinylalkyl, piperazinylalkyl, cyclohexylalkyl, indolyl, 2,3-dihydroindolyl, 1-acetyl-2,3-dihydroindolyl, cycloheptyl, bicyclo[2.2.1]hept-2-yl, hydroxyphenyl, hydroxyalkylphenyl, pyrrolidinyl, pyrrolidin-1-yl, pyrrolidin-1-ylalkyl, 4-amino(imino)methylphenyl, isoxazolyl, indazolyl, adamantyl, bicyclohexyl, quinuclidinyl, imidazolyl, benzimidazolyl, imidazolylphenyl, phenylimidazolyl, pthalamido, napthyl, benzophenone, anilinyl, anisoyl, quinolinyl, quinolinonyl, phenylsulfonyl, phenylalkylsulfonyl, 9H-flouren-1-yl, piperidin-1-yl, piperidin-1-ylalkyl, cyclopropyl, cyclopropylalkyl, pyrimidin-5-ylphenyl, quinolidinylphenyl, furanyl, furanylphenyl, N-methylpiperidin-4-yl, pyrrolidin-4-ylpyridinyl, 4-diazepan-1-yl, hydroxypyrrolidin-1-yl, dialkylaminopyrrolidin-1-yl, 1,4′-bipiperidin-1′-yl, and (1,4′-bipiperidin-1′-ylcarbonyl)phenyl;
A₂ is substituted or unsubstituted pyridyl;
R₁ is O and the dotted line represents a single or double bond;
R₂ is NR₅R₆, R₅ is hydrogen and R₆ is selected from hydrogen and substituted or unsubstituted alkyl, all; oxyalkyl, aminoalkyl, amidoalkyl, acyl, cycloalkyl, heterocycloalkyl, aryl, heteroaryl, alkyloxyalkylheterocyclo, and heteroarylalkyl:
R₁ and R₂ together with the carbon atom to which they are attached form a substituted or unsubstituted heterocycloalkyl or heteroaryl group.
R₃ is loweralkoxy, e.g. methoxy;
R₄ is loweralkyl; e.g. methyl;
R₁ is O, R₂ is NR₅R₆, R₅ is H. and R₆ is methyl;
   e.g. including a compound of formula I_{WO03082272/WO2005032548}, which is a compound of formula

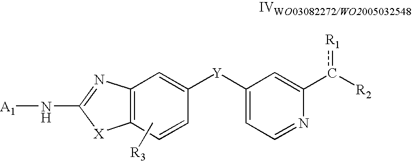

wherein

### X is NR₄, O or S;

### Y is O or S.;

A₁ is substituted or unsubstituted cycloalkyl, heterocycloalkyl, aryl, polycyclic aryl, polycyclic arylalkyl, heteroaryl, biaryl, heteroarylaryl, heteroarylheteroaryl, cycloalkylalkyl, heterocycloalkylalkyl, arylalkyl, heteroarylalkyl, biarylalkyl, heteroarylarylalkyl;

### R₁ is O and R₂ is NR₅R₆; or

R₁ and R₂ together with the carbon atom to which they are attached form a substituted or unsubstituted heterocycloalkyl or heteroaryl group; wherein, the dotted line represents a single or double bond;
R₃ is hydrogen, halogen, loweralkyl, or loweralkoxy;
R₄ is hydrogen or loweralkyl;
R₅ and R₆ independently of each other are selected from hydrogen substituted or unsubstituted alkyl, alkoxyalkyl, aminoalkyl, amidoalkyl, acyl, cycloalkyl, heterocycloalkyl, aryl, heteroaryl, alkyloxyalkylheterocyclo, and heteroarylalkyl; or
R₅ and R₆ together with the nitrogen atom to which they are attached form substituted or unsubstituted heterocyclo or heteroaryl; or
A pharmaceutically acceptable salt, ester, or prodrug thereof;
e.g. including a compound of formula I_{WO03082272/WO2005032548}, wherein
X is NR₄;
R₄ is hydrogen;
R₄ is methyl;
Y is O.
A₁ is selected from the group consisting of substituted or unsubstituted phenyl, pyridyl, pyrimidinyl, phenylalkyl, pyridylalkyl, pyrimidinylalkyl, heterocyclylcarbonylphenyl, heterocyclylphenyl, heterocyclylalkylphenyl, chlorophenyl, fluorophenyl, bromophenyl, iodophenyl, dihalophenyl, nitrophenyl, 4-bromophenyl, 4-chlorophenyl, alkylbenzoate, alkoxyphenyl, dialkoxyphenyl, dialkylphenyl, trialkylphenyl, thiophene, thiophene-2-carboxylate, alkylthiophenyl, trifluoromethylphenyl, acetylphenyl, sulfamoylphenyl, biphenyl, cyclohexylphenyl, phenyloxyphenyl, dialkylaminophenyl, alkylbromophenyl, alkylchlorophenyl, alkylfluorophenyl, trifluoromethylchlorophenyl, trifluoromethylbromophenyl indenyl, 2,3-dihydroindenyl, tetralinyl, trifluorophenyl, (trifluoromethyl)thiophenyl, alkoxybiphenyl, morpholinyl, N-piperazinyl? N-morpholinylalkyl, piperazinylalkyl, cyclohexylalkyl, indolyl, 2,3-dihydroindolyl, 1-acetyl-2,3-dihydroindolyl? cycloheptyl, bicyclo[2.2.1]hept-2-yl, hydroxyphenyl, hydroxyalkylphenyl, pyrrolidinyl, pyrrolidin-1-yl, pyrrolidin-1-ylalkyl, 4-amino(imino)methylphenyl, isoxazolyl, indazolyl, adamantyl, bicyclohexyl, quinuclidinyl, imidazolyl, benzimidazolyl2 imidazolylphenyl, phenylimidazolyl, pthalamido, napthyl, benzophenone, anilinyl, anisoyl, quinolinyl, quinolinonyl, phenylsulfonyl, phenylalkylsulfonyl, 9H-flouren-1-yl, piperidin-1-yl, piperidin-1-yla1ky1, cyclopropyl, cyclopropylalkyl, pyrimidin-5-ylphenyl, quinolidinylphenyl, furanyl, furanylphenyl, N-methylpiperidin-4-yl, pyrrolidin-4-ylpyridinyl, 4-diazepan-1-yl, hydroxypyrrolidin-1-yl, dialkylaminopyrrolidin-1-yl, 1,4′-bipiperidin-1′-yl, and (1,4′-bipiperidin-1′-ylcarbonyl)phenyl;
R₁ is O and the dotted line represents a single or double bond;
R₂ is NR₅R₆, R₅ is hydrogen and R₆ is selected from hydrogen and substituted or unsubstituted alkyl, alkoxyalkyl, aminoalkyl, amidoalkyl, acyl, cycloalkyl, heterocycloalkyl, aryl, heteroaryl, alkyloxyalkylheterocyclo, and heteroarylalkyl;
R₁ and R₂ together with the carbon atom to which they are attached form a substituted or unsubstituted heterocycloalkyl or heteroaryl group;
R₃ is loweralkoxy, e.g. methoxy;
R₄ is loweralkyl, e.g. methyl;
R₁ is O, R₂ is NR₅R₆, R₅ is H. and R₆ is methyl;
   e.g. including a compound of formula I_{WO03082272/WO2005032548}, which is a compound of formula

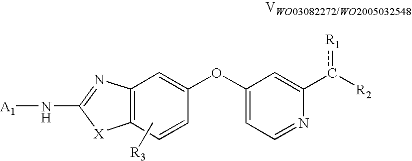

wherein

### X is NR₄, O or S;

A₁ is substituted or unsubstituted cycloalkyl, heterocycloalkyl, aryl, polycyclic aryl, polycyclic arylalkyl, heteroaryl, biaryl, heteroarylaryl, heteroarylheteroaryl, cycloalkylalkyl, heterocycloalkylalkyl, arylalkyl, heteroarylalkyl, biarylalkyl, heteroarylarylalkyl;

### R₁ is O and R₂ is NR₅; or

R₁ and R₂ together with the carbon atom to which they are attached form a substituted or unsubstituted heterocycloalkyl or heteroaryl group; wherein, the dotted line represents a single or double bond;
R₃ is hydrogen, halogen, loweralkyl, or loweralkoxy;
R₄ is hydrogen or loweralkyl;
R₅ and R₆ are independently selected from hydrogen, and substituted or unsubstituted alkyl, alkoxyalkyl, aminoalkyl, amidoalkyl, acyl, cycloalkyl, heterocycloalkyl, aryl, heteroaryl, alkyloxyalkylheterocyclyl, and heteroarylalkyl; or
R₅ and R₆ are taken together to form substituted or unsubstituted heterocyclyl or heteroaryl; or
a pharmaceutically acceptable salt, ester or prodrugs thereof;
e.g. including a compound of formula V_{WO03082272/WO2005032548}, wherein
X is NR₄.
R₄ is hydrogen.
R₄ is methyl.
A₁ is selected from the group consisting of substituted or unsubstituted phenyl, pyridyl, pyrimidinyl, phenylalkyl, pyridylalkyl, pyrimidinylalkyl, heterocyclylcarbonylphenyl, heterocyclylphenyl, heterocyclylalkylphenyl, chlorophenyl, fluorophenyl, bromophenyl, iodophenyl, dihalophenyl, nitrophenyl, 4-bromophenyl, 4-chlorophenyl, alkylbenzoate, alkoxyphenyl, dialkoxyphenyl, dialkylphenyl, trialkylphenyl, thiophene, thiophene-2-carboxylate, alkylthiophenyl, trifluoromethylphenyl, acetylphenyl, sulfamoylphenyl, biphenyl, cyclohexylphenyl, phenyloxyphenyl, dialkylaminophenyl, alkylbromophenyl, alkylchlorophenyl, alkylfluorophenyl, trifluoromethylchlorophenyl, trifluoromethylbromophenyl indenyl, 2,3-dihydroindenyl, tetralinyl, trifluorophenyl, (trifluoromethyl)thiophenyl, alkoxybiphenyl, morpholinyl, N-piperazinyl, N-morpholinylalkyl, piperazinylalkyl, cyclohexylalkyl, indolyl, 2,3-dihydroindolyl, 1-acetyl-2,3-dihydroindolyl, cycloheptyl, bicyclo[2.2.1]hept-2-yl, hydroxyphenyl, hydroxyalkylphenyl, pyrrolidinyl, pyrrolidin-1-yl, pyrrolidin-1-ylalkyl, 4-amino(imino)methylphenyl, isoxazolyl, indazolyl, adamantyl, bicyclohexyl, quinuclidinyl, imidazolyl, benzimidazolyl, imidazolylphenyl, phenylimidazolyl, pthalamido, napthyl, benzophenone, anilinyl, anisoyl, quinolinyl, quinolinonyl, phenylsulfonyl, phenylalkylsulfonyl, 9H-flouren-1-yl, piperidin-1-yl, piperidin-1-ylalkyl, cyclopropyl, cyclopropylalkyl, pyrimidin-5-ylphenyl, quinolidinylphenyl, furanyl, furanylphenyl, N-methylpiperidin-4-yl, pyrrolidin-4-ylpyridinyl, 4-diazepan-1-yl, hydroxypyrrolidin-1-yl, dialkylaminopyrrolidin-1-yl, 1,4′-bipiperidin-1′-yl, and (1,4′-bipiperidin-1′-ylcarbonyl)phenyl;
R₁ is O and the dotted line represents a single or double bond;
R₂ is NR₅R₆, R₅ is hydrogen and R₆ is selected from hydrogen, and substituted or unsubstituted alkyl, alkoxyalkyl, aminoalkyl, amidoalkyl, acyl, cycloalkyl, heterocycloalkyl, aryl, heteroaryl, alkyloxyalkylheterocyclo, and heteroarylalkyl;
R₁ and R₂ to form a substituted or unsubstituted heterocycloalkyl or heteroaryl group;
R₃ is loweralkoxy, e.g. methoxy;
R₄ is loweralkyl, e.g. methyl;
R₁ is O, R₂ is NR₅R₆, R₅ is H. and R₆ is methyl.
   e.g. a compound of formula I_{WO03082272/WO2005032548}, which is selected from the group consisting of the compounds of
   the compound of example 1, the compounds of Table 1 (compounds 2 to 108), the compound of example 109, the compounds of Table 2 (compounds 110 to 119), the compounds of Examples 120 (a+b), the compounds of Table 3 (compounds 121 to 371), the compound of example 372, the compounds of Table 4 (compounds 373 to 448), the compounds of examples 450 to 451, the compounds of Table 5 (compounds 452 to 481), the compounds of examples 482 to 489, the compounds of Table 6 (compounds 490 to 626), the compounds of examples 627 to 638, the compounds of Table 7 (compounds 639 to 698), the compounds of examples 699 to 704, the compounds of Table 8 (compounds 705 to 746), the compounds of Table 9 (compounds 747 to 782), the compounds of examples 783 to 784, the compounds of Table 10 (compounds 785 to 802), the compound of example 803, the compounds of Table 11 (compounds 804 to 812), the compounds of examples 813 to 815, the compounds of Table 12 (compounds 816 to 819), the compounds of examples 820 to 822, the compounds of Table 13 (compounds 823 to 984), the compounds of examples 985 to 1036, the compounds of Table 14 (compounds 1037 to 1094b), and the compounds of examples 1095 to 1115; of WO03082272 and/or WO2005032548, and the compounds of examples 1116 to 1163 and the compounds of Table 16 (compounds 1164 to 1400) of WO2005032548.
   e.g. or
   a pharmaceutically acceptable salt, ester or prodrug thereof;
   as disclosed in WO03082272 and/or in WO2005032548;
   e.g. wherein each single compound listed may be a preferred compound.

Preferred meanings of substituents of a compound of formula I_{WO03082272/WO2005032548}, II_{WO03082272/WO2005032548}, III_{WO03082272/WO2005032548}, IV_{WO03082272/WO2005032548}, or of formula V_{WO03082272/WO2005032548}, e.g. including the meaning of the substituents X, X₁, X₂, Y, A₁, A₂, R₁, R₂, R₃, R₄, R₅ and R₆, as indicated herein, are as defined in WO03082272 and/or in WO2005032548, and it is referred herein to WO03082272 and WO2005032548; and the content of WO03082272 and of WO2005032548 is introduced herein by reference.

The compounds of WO03082272 and/or WO2005032548 may be administered, e.g. in the form of a pharmaceutical composition as described in WO03082272 or WO2005032548. A therapeutically effective dose will generally be a total daily dose administered to a subject in need thereof in single or divided doses, may be in amounts, for example, of from 0.001 to 1000 mg/kg body weight daily and more preferred from 1.0 to 30 mg/kg body weight daily. Dosage unit compositions may contain such amounts of submultiples thereof to make up the daily dose.

It is referred herein to WO03082272 and WO2005032548 in any aspect; and the content of WO03082272 and of WO2005032548 is introduced herein by reference.

In another aspect the present invention provides a combination of an mTOR inhibitor with a compound as disclosed in WO2007030377 which is a compound of formula

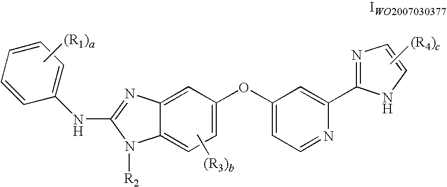

wherein
each R₁ is independently selected from hydroxy, halo, C₁₋₆alkyl, C₁₋₆alkoxy, (C₁₋₆alkyl)sulfanyl, (C₁₋₆alkyl)sulfonyl, cycloalkyl, heterocycloalkyl, phenyl, and heteroaryl;
R₂ is C₁₋₆alkyl or halo(C₁₋₆alkyl);
each R₃ is independently selected from halo, C₁₋₆alkyl, and C₁₋₆alkoxy;
each R₄ is independently selected from hydroxy, C₁₋₆alkyl, C₁₋₆alkoxy, halo, carboxyl, (C₁₋₆alkoxy)carbonyl, aminocarbonyl, C₁₋₆alkylaminocarbonyl, carbonitrile, cycloalkyl, heterocycloalkyl, heterocycloalkylcarbonyl, phenyl, and heteroaryl;
wherein R₁, R₂, R₃, and R₄ may be optionally substituted with one or more substituents independently selected from hydroxy, halo, C₁₋₆alkyl, halo(C₁₋₆alkyl), C₁₋₆alkoxy, and halo(C₁₋₆alkoxy);
a is 1, 2, 3, 4, or 5;
b is 0, 1, 2, or 3; and
c is 1 or 2;
or a tautomer, stereoisomer, polymorph, ester, metabolite, or prodrug thereof or a pharmaceutically acceptable salt of the compound, tautomer, stereoisomer, polymorph, ester, metabolite, or prodrug,
e.g. including a compound of formula I_{WO2007030377} wherein
each R₁ is independently selected from the group consisting of hydroxy, chloro, fluoro, bromo, methyl, ethyl, propyl, butyl, methoxy, ethoxy, propoxy, butoxy, trifluoromethyl, trifluoroethyl, trifluoromethoxy, trifluoroethoxy, piperidinyl, C₁₋₆alkylpiperidinyl, piperazinyl, C₁₋₆alkylpiperazinyl, tetrahydrofuranyl, pyridinyl, and pyrimidinyl;
a is 1 or 2, and at least one R₁ is halo(C₁₋₆alkyl).
at least one R₂ is trifluoromethyl;
R₂ is C₁₋₆ alkyl, e.g. methyl or ethyl, such as methyl.
b is 0, and R₃ is not present;
b is 1, and R₃ is C₁₋₆alkoxy; e.g. methoxy;
c is 1 or 2, and at least one R₄ is halo(C₁₋₆alkyl), such as trifluoromethyl;
   e.g. including a compound of formula I_{WO2007030377}, which is a compound of formula

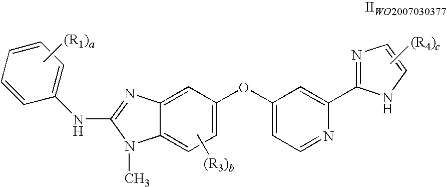

wherein
each R₁ is independently selected from C₁₋₆alkyl, C₁₋₆alkoxy, hydroxy, halo (C₁₋₆alkyl)sulfanyl, (C₁₋₆alkyl)sulfonyl, cycloalkyl, heterocycloalkyl, phenyl, and heteroaryl;
each R₃ is independently selected from haloC₁₋₆alkyl and C₁₋₆alkoxy;
each R₄ is independently selected from hydroxy, C₁₋₆alkyl, C₁₋₆alkoxy, halo, carboxyl, (C₁₋₆alkoxy)carbonyl, aminocarbonyl, carbonitrile, cycloalkyl, heterocycloalkyl, heterocycloalkylcarbonyl, phenyl, and heteroaryl;
wherein R₁, R₂, R₃, and R₄ is optionally substituted with one or more substituents independently selected from hydroxy, halo, C₁₋₆alkyl and C₁₋₆alkoxy;
a is 1, 2, 3, 4 or 5;
b is 0, 1, 2, or 3; and
c is 1 or 2; or
a tautomer, stereoisomer, polymorph, ester, metabolite, or prodrug thereof or a pharmaceutically acceptable salt of the compound, tautomer, stereoisomer, polymorph, ester, metabolite, or prodrug;
e.g. including a compound of formula I_{WO2007030377} or of formula II_{WO2007030377}, which is a compound of formula

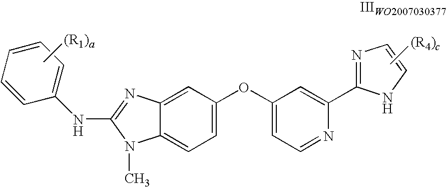

wherein,
each R₁ is independently selected from C₁₋₆alkyl, C₁₋₆alkoxy, hydroxy, halo, (C₁₋₆alkyl)sulfanyl, (C₁₋₆alkyl)sulfonyl, cycloalkyl, heterocycloalkyl, phenyl, and heteroaryl;
each R₄ is independently selected from hydroxy, C₁₋₆alkyl, C₁₋₆alkoxy, halo, carboxyl, (C₁₋₆alkoxy)carbonyl, aminocarbonyl, carbonitrile, cycloalkyl, heterocycloalkyl, heterocycloalkylcarbonyl, phenyl, and heteroaryl;
wherein R₁ and R₄ may be optionally substituted with one or more substituents independently selected from hydroxy, halo, C₁₋₆alkyl and C₁₋₆alkoxy,
a is 1, 2, 3, 4, or 5; and
c is 1 or 2;
or a tautomer, stereoisomer, polymorph, ester, metabolite, or prodrug thereof, or a pharmaceutically acceptable salt of the compound, tautomer, stereoisomer, polymorph, ester, metabolite, or prodrug;
e.g. including a compound of formula III_{WO2007030377} wherein
each R₁ is independently selected from the group consisting of hydroxy, chloro, fluoro, bromo, methyl, ethyl, propyl, butyl, methoxy, ethoxy, propoxy, butoxy, trifluoromethyl, trifluoroethyl, trifluoromethoxy, trifluoroethoxy, piperidinyl, C₁₋₆alkylpiperidinyl, piperazinyl, C₁₆ alkylpiperazinyl, tetrahydrofuranyl, pyridinyl and pyrimidinyl; e.g. and a is 1 or 2, and at least one R₁ is halo(C₁₋₆alkyl); e.g. R₁ is trifluoromethyl;
each R₁ is independently selected from the group consisting of hydroxy, chloro, fluoro, bromo, methyl, ethyl, propyl, butyl, methoxy, ethoxy, propoxy, butoxy, trifluoromethyl, trifluoroethyl, trifluoromethoxy, trifluoroethoxy, piperidinyl, C₁₋₆alkylpiperidinyl, piperazinyl, C₁₋₆alkylpiperazinyl, tetrahydrofuranyl, pyridinyl and pyrimidinyl and a is 1; e.g. and R₁ is trifluoromethyl;
each R₁ is independently selected from the group consisting of hydroxy, chloro, fluoro, bromo, methyl, ethyl, propyl, butyl, methoxy, ethoxy, propoxy, butoxy, trifluoromethyl, trifluoroethyl, trifluoromethoxy, trifluoroethoxy, piperidinyl, C₁₋₆alkylpiperidinyl, piperazinyl, C₁₋₆ alkylpiperazinyl, tetrahydrofuranyl, pyridinyl and pyrimidinyl and c is 1 or 2, such as 1, and at least one R₄ is halo(C₁₋₆alkyl), such as trifluoromethyl;
   e.g. or a tautomer, stereoisomer, polymorph, ester, metabolite, or prodrug thereof, or a pharmaceutically acceptable salt of the compound, tautomer, stereoisomer, polymorph, ester, metabolite, or prodrug;
   e.g. including a compound of formula I_{WO2007030377}, which is selected from the group consisting of
{1-Methyl-5-[2-(5-trifluoromethyl-1H-imidazol-2-yl)-pyridin-4-yloxyl]-1H-benzo-imidazol-2-yl}-(4-trifluoromethylphenyl)amine,
(2-Fluoro-5-pyridin-3-yl-phenyl)-{1-methyl-5-[2-(5-trifluoromethyl-1H-imidazol-2-yl)-pyridin-4-yloxy]-1H-benzoimidazol-2-yl}-amine,
(2-Fluoro-5-pyridin-4-yl-phenyl)-{1-methyl-5-[2-(5-trifluoromethyl-1H-imidazol-2-yl)-pyridin-4-yloxy]-1H-benzoimidazol-2-yl}-amine,
(4-tert-Butyl-phenyl)-{1-methyl-5-[2-(5-trifluoromethyl-1H-imidazol-2-yl)-pyridin-4-yloxy]-1H-benzoimidazol-2-yl}-amine,
{1-Methyl-5-[2-(5-trifluoromethyl-1H-imidazol-2-yl)pyridin-4-yloxy]-1H-benzo-imidazol-2-yl}-(3-trifluoromethyl-phenyl)-amine,
(3-Ethyl-phenyl)-{1-methyl-5-[2-(5-trifluoromethyl-1H-imidazol-2-yl)-pyridin-4-yl-oxy]-1H-benzoimidazol-2-yl}-amine,
(4-Chloro-phenyl)-{1-methyl-5-[2-(5-trifluoromethyl-1H-imidazol-2-yl)-pyridin-4-yloxy]-1H-benzoimidazol-2-yl}-amine,
(4-Ethyl-phenyl)-{1-methyl-5-[2-(5-trifluoromethyl-1H-imidazol-2-yl)-pyridin-4-yl-oxy]-1H-benzoimidazol-2-yl}-amine,
(4-Chloro-3-trifluoromethyl-phenyl)-{1-methyl-5-{2-(5-trifluoromethyl-1H-imidazol-2-yl)-pyridin-4-yloxy}-1H-benzoimidazol-2-yl}-amine,
(4-Fluoro-3-trifluoromethyl-phenyl)-{1-methyl-5-[2-(5-trifluoromethyl-1H-imidazol-2-yl)-pyridin-4-yloxy]-1H-benzoimidazol-2-yl}-amine,
{1-Methyl-5-[2-(5-trifluoromethyl-1H-imidazol-2-yl)-pyridin-4-yloxy]-1H-benzo-imidazol-2-yl}-(4-trifluoromethoxy-phenyl)-amine,
(2-Fluoro-5-trifluoromethyl-phenyl)-(1-methyl-5-{2-[5-methyl-4-(3-trifluoromethyl-phenyl)-1H-imidazol-2-yl]-pyridin-4-yloxy}1H-benzoimidazol-2-yl)-amine,
(2-Fluoro-5-trifluoromethyl-phenyl)-(1-methyl-5-{2-{-5-methyl-4-(4-trifluoromethyl-phenyl)-1H-imidazol-2-yl]-pyridin-4-yloxy}-1H-benzoimidazol-2-yl)amine,
2-{4-[2-(2-Fluoro-5-trifluoromethyl-phenylamino)-1-methyl-1H-benzoimidazol-5-yloxy]-pyridin-2-yl}-5-trifluoromethyl-1H-imidazole-4-carboxylic acid ethyl ester,
(2-{4-[2-(2-Fluoro-5-trifluoromethyl-phenylamino)-1-methyl-1H-benzoimidazol-5-yloxy]-pyridin-2-yl}-5-trifluoromethyl-1H-imidazol-4-yl)-methanol,
2-{4-[1-Methyl-2-(4-trifluoromethyl-phenylamino)-1H-benzoimidazol-5-yloxy]-pyridin-2-yl}-3H-imidazole-4-carbonitrile,
(3-tert-Butyl-phenyl)-{1-methyl-5-[2-(5-phenyl-1H-imidazol-2-yl)pyridin-4-yl-oxy]-1H-benzoimidazol-2-yl}-amine,
{{1-Methyl-5-[2-(5-phenyl-1H-imidazol-2-yl)pyridine-4-yloxy]-1H-benzoimidazol-2-yl}(4-trifluoromethylsulfanyl-phenyl)-amine,
(3-tert-Butyl-phenyl)-{1-methyl-5-[2-(5-trifluoromethyl-1H-imidazol-2-yl)pyridin-4-yloxy}-1H-benzoimidazol-2-yl]amine,
[4-Fluoro-3-(tetrahydro-furan-3-yl)phenyl]-{1-methyl-5-{2-(5-trifluoromethyl-1H-imidazol-2-yl)-pyridin-4-yloxy]-1H-benzoimidazol-2-yl}amine,
(4-Bromo-phenyl)-{1-methyl-5-[2-(5-trifluoromethyl-1H-imidazol-2-yl)-pyridin-4-yloxy]-1H-benzoimidazol-2-yl}-amine,
(4-Fluoro-3-isopropyl-phenyl)-{1-methyl-5-[2-(5-trifluoromethyl-1H-imidazol-2-yl)-pyridin-4-yloxy}1H-benzoimidazol-2-yl]amine,
{1-Methyl-5-[2-(5-trifluoromethyl-1H-imidazol-2-yl)-pyridin-4-yloxy}-1H-benzoimidazol-2-yl](4-trifluoromethylsulfanyl-phenyl)-amine,
(2-Fluoro-5-isopropyl-phenyl)-{1-methyl-5-[2-(5-trifluoromethyl-1H-imidazol-2-yl)-pyridin-4-yloxy]-1H-benzoimidazol-2-yl}-amine,
(2-Fluoro-5-trifluoromethyl-phenyl)-{1-methyl-5-[2-(5-trifluoromethyl-1H-imidazol-2-yl) pyridin-4-yloxy]-1H-benzoimidazol-2-yl}-amine,
(5-tert-Butyl-2-fluoro-phenyl)-{1-methyl-5-[2-(5-trifluoromethyl-1H-imidazol-2-yl)-pyridin-4-yloxy]-1H-benzoimidazol-2-yl}-amine,
(2-Fluoro-5-trifluoromethyl-phenyl)-{1-methyl-5-[2-(5-methyl-1H-imidazol-2-yl)-pyridin-4-yloxy]-1H-benzoimidazol-2-yl}-amine,
(2-Fluoro-5-pyridin-3-yl-phenyl)-{1-methyl-5-[2-(5-trifluoromethyl-1H-imidazol-2-yl)-pyridin-4-yloxy]-1H-benzoimidazol-2-yl}-amine,
2-{4-[2-(2-Fluoro-5-trifluoromethyl-phenylamino)-1-methyl-1H-benzoimidazol-5-yloxy]-pyridin-2-yl}-3H-imidazole-4-carbonitrile,
(2-Chloro-4-trifluoromethyl-phenyl)-{1-methyl-5-[2-(5-trifluoromethyl-1H-imidazol-2-yl)-pyridin-4-yloxy]-1H-benzoimidazol-2-yl}amine,
(5-tert-Butyl-2-chloro-phenyl)-{1-methyl-5-[2-(5-trifluoromethyl-1H-imidazol-2-yl)-pyridin-4-yloxy]-1H-benzoimidazol-2-yl}-amine,
(2-Fluoro-5-pyridin-4-yl-phenyl)-{1-methyl-5-{2-(5-trifluoromethyl-1H-imidazol-2-yl)pyridin-4-yloxy]-1H-benzoimidazol-2-yl}-amine,
(2-Fluoro-5-trifluoromethyl-phenyl)-{1-methyl-5-{2-(4-phenyl-5-trifluoromethyl-1H-imidazol-2-yl)-pyridin-4-yloxy]-1H-benzoimidazol-2-yl}-amine,
(2-Chloro-5-trifluoromethyl-phenyl)-{1-methyl-5-[2-(4-phenyl-5-trifluoromethyl-1H-imidazol-2-yl)-pyridin-4-yloxy]-1H-benzoimidazol-2-yl}-amine,
{1-Methyl-5-[2-(4-phenyl-5-trifluoromethyl-1H-imidazol-2-yl)-pyridin-4-yloxy]-1H-benzoimidazol-2-yl}-(3-trifluoromethyl-phenyl)-amine,
(3-Ethyl-phenyl)-{1-methyl-5-[2-(4-phenyl-5-trifluoromethyl-1H-imidazol-2-yl)-pyridin-4-yloxy]-1H-benzoimidazol-2-yl}-amine,
(4-tert-Butyl-phenyl)-{1-methyl-5-[2-(4-phenyl-5-trifluoromethyl-1H-imidazol-2-yl)-pyridin-4-yloxy]-1H-benzoimidazol-2-yl}-amine,
(2-Chloro-5-trifluoromethyl-phenyl)-{1-methyl-5-[2-(5-trifluoromethyl-1H-imidazol-2-yl)-pyridin-4-yloxy]-1H-benzoimidazol-2-yl}amine,
(2-Fluoro-5-trifluoromethyl-phenyl)-{1-methyl-5-[2-(5-methyl-4-phenyl-1H-imidazol-2-yl) pyridin-4-yloxy]-1H-benzoimidazol-2-yl}amine,
(2-Chloro-5-trifluoromethyl-phenyl)-{1-methyl-5-[2-(5-methyl-4-phenyl-1H-imidazol-2-yl)-pyridin-4-yloxy]-1H-benzoimidazol-2-yl}amine,
(4-tert-Butyl-phenyl)-{1-methyl-5-[2-(5-methyl-4-phenyl-1H-imidazol-2-yl)-pyridin-4-yloxy]-1H-benzoimidazol-2-yl}amine,
{1-Methyl-5-{2-(5-methyl-4-phenyl-1H-imidazol-2-yl)-pyridin-4-yloxy]-1H-benzo-imidazol-2-yl}-(3-trifluoromethyl-phenyl)-amine,
(5-tert-Butyl-2-fluoro-phenyl)-{1-methyl-5-[2-(5-methyl-4-phenyl-1H-imidazol-2-yl)-pyridin-4-yloxy]-1H-benzoimidazol-2-yl}-amine,
[4-(4-Methyl-piperazin-1-yl)-phenyl]-{-1-methyl-5-[2-(5-trifluoromethyl-1H-imidazol-2-yl)-pyridin-4-yloxy]-1H-benzoimidazol-2-yl}-amine,
2-{4-[2-(2-Fluoro-5-trifluoromethyl-phenylamino)-1-methyl-1H-benzoimidazol-5-yloxy]-pyridin-2-yl}-3H-imidazole-4-carboxylic acid methyl ester,
2-{4-[2-(2-Chloro-5-trifluoromethyl-phenylamino)-1-methyl-1H-benzoimidazol-5-yloxy]-pyridin-2-yl}-5-trifluoromethyl-1H-imidazole-4-carboxylic acid ethyl ester,
(2-Fluoro-4-trifluoromethyl-phenyl)-{1-methyl-5-2-(5-trifluoromethyl-1H-imidazol-2-yl)-pyridin-4-yloxy]-1H-benzoimidazol-2-yl}-amine,
(2-Chloro-phenyl)-{1-methyl-5-{2-(5-trifluoromethyl-1H-imidazol-2-yl)-pyridin-4-yloxy]-1H-benzoimidazol-2-yl}-amine,
(2,5-Dimethoxy-phenyl)-{1-methyl-5-{2-(5-trifluoromethyl-1H-imidazol-2-yl)-pyridin-4-yloxy]-1H-benzoimidazol-2-yl}amine,
(3,5-Dimethoxy-phenyl)-{1-methyl-5-[2-(5-trifluoromethyl-1H-imidazol-2-yl)-pyridin-4-yloxy]-1H-benzoimidazol-2-yl}-amine,
{1-Methyl-5-{2-(5-trifluoromethyl-1H-imidazol-2-yl)pyridin-4-yloxy}-1H-benzoimidazol-2-yl}-(2-trifluoromethyl-phenyl)-amine,
(2-Ethyl-phenyl)-{1-methyl-5-[2-(5-trifluoromethyl-1H-imidazol-2-yl)pyridin-4-yl-oxy]-1H-benzoimidazol-2-yl}amine,
(4-Ethyl-piperazin-1-yl)-(2-{4-[2-(2-fluoro-5-trifluoromethyl-phenylamino)-1-methyl-1H-benzoimidazol-5-yloxy]-pyridin-2-yl}-3H-imidazol-4-yl)-methanone,
2-{4-{2-(2-Fluoro-5-trifluoromethyl-phenylamino)-1-methyl-1H-benzoimidazol-5-yloxy]-pyridin-2-yl}-3H-imidazole-4-carboxylic acid (2-hydroxy-ethyl)-amide,
{1-Ethyl-5-[2-(5-trifluoromethyl-1H-imidazol-2-yl)pyridin-4-yloxy]-1H-benzoimidazol-2-yl}(2-fluoro-5-trifluoromethyl-phenyl)-amine,
(2-Fluoro-5-trifluoromethyl-phenyl)-{6-methoxy-1-methyl-5-[2-(5-trifluoromethyl-1H-imidazol-2-yl)-pyridin-4-yloxy]-1H-benzoimidazol-2-yl}amine,
{6-Methoxy-1-methyl-5-2-(5-trifluoromethyl-1H-imidazol-2-yl)-pyridin-4-yloxy]-1H-benzoimidazol-2-yl}-(4-trifluoromethyl-phenyl)-amine,
(4-Ethyl-piperazin-1-yl)-(2-{4-[1-methyl-2-(4-trifluoromethyl-phenylamino)-1H-benzoimidazol-5-yloxy]-pyridin-2-yl}-3H-imidazol-4-yl)-methanone,
{1-Ethyl-5-[2-(5-trifluoromethyl-1H-imidazol-2-yl)-pyridin-4-yloxy]-1H-benzoimidazol-2-yl}(4-trifluoromethyl-phenyl)-amine,
2-{4-{1-Methyl-2-(4-trifluoromethyl-phenylamino)-1H-benzoimidazol-5-yloxy]-pyridin-2-yl}-3H-imidazole-4-carboxylic acid (2-hydroxy-ethyl)-amide,
2-{1-Methyl-5-[2-(5-trifluoromethyl-1H-imidazol-2-yl)-pyridin-4-yloxy]-1H-benzoimidazol-2-ylamino}-5-trifluoromethyl-phenol, and
3-{1-Methyl-5-{2-(5-trifluoromethyl-1H-imidazol-2-yl)-pyridin-4-yloxy]-1H-benzoimidazol-2-ylamino}-6-trifluoromethyl-phenol;
   or a tautomer, stereoisomer, polymorph, ester, metabolite, or prodrug thereof or a pharmaceutically acceptable salt of the compound, tautomer, stereoisomer, polymorph, ester, metabolite, or prodrug;
   such as a compound of formula I_{WO2007030377} which is the compound

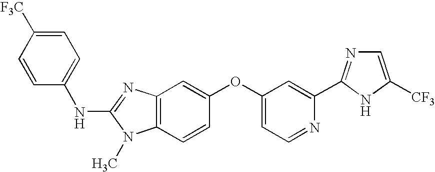

or a pharmaceutically acceptable salt thereof,
or a tautomer thereof of formula

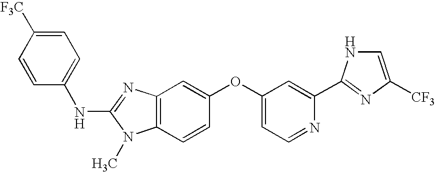

or a pharmaceutically acceptable salt thereof;
such as a compound as disclosed in examples 1 to 16, Table 1 (compounds 17 to 59a), example 60, Table 2 (compounds 61 to 64), and in examples 73 to 75 in WO2007030377.

Preferred meanings of substituents of a compound of formula I_{WO2007030377}, II_{WO2007030377}, or of formula III_{WO2007030377}, e.g. including the meaning of the substituents R₁, R₂, R₃, R₄, a, b and c, as indicated herein, are as defined in WO2007030377, and it is referred herein to WO2007030377; and the content of WO2007030377 is introduced herein by reference.

The compounds of WO2007030377 may be administered, e.g. in the form of a pharmaceutical composition as described in WO2007030377. A therapeutically effective dose will generally be a total daily dose administered to a subject in need thereof in single or divided doses, may be in amounts, for example, of from 0.001 to 1000 mg/kg body weight daily and more preferred from 1.0 to 30 mg/kg body weight daily. Dosage unit compositions may contain such amounts of submultiples thereof to make up the daily dose. It is referred herein to WO2007030377 in any aspect; and the content of WO2007030377 is introduced herein by reference.

A combination of the present invention is useful for the treatment of cancer, e.g. including all types of cancer. Preferably a combination of the present invention may be used for the treatment of tumors, e.g. carcinoma, including that of the bladder, breast, colon, kidney, liver, lung, ovary, pancreas, stomach, cervix, thyroid and skin; -hematopoietic tumors of lymphoid lineage, including acute lymphocytic leukemia, B-cell lymphoma and Burketts lymphoma; -hematopoietic tumors of myeloid lineage, including acute and chronic myelogenous leukemias and promyelocytic leukemia; tumors of mesenchymal origin, including fibrosarcoma and rhabdomyosarcoma; and-other tumors, including melanoma, seminoma, teratocarcinoma, neuroblastoma and glioma.

A glioma is a type of primary central nervous system (CNS) tumor that arises from glial cells. The most common site of involvement of a glioma is the brain, but they can also affect the spinal cord, or any other part of the CNS, such as the optic nerves. Types of gliomas e.g. include
astrocytomas, which start in brain cells called astrocytes and can occur in most parts of the brain (and occasionally in the spinal cord; they are most commonly found in the main part of the brain, the cerebrum;
ependymomas, which begin in the ependymal, the cells that line the passageways in the brain where special fluid that protects the brain and spinal cord (called cerebrospinal fluid) is made and stored. They are a rare glioma and can be found anywhere in the brain or spine, but most commonly in the main part of the brain, the cerebrum:
oligodendrogliomas, which are primary brain tumors beginning in the brain cells called oligodendrocytes, which provide support and nourishment for the cells that transmit nerve impulses. This tumor is normally found in the cerebrum;
mixed gliomas, which are brain tumors of more than one type of brain cell, including cells of astrocytes, ependymal cells and/or oligodendrocytes. The most common site for a mixed glioma is the cerebrum, the main part of the brain. Like other gliomas, they may spread to other parts of the brain.

Low grade gliomas are slow growing. High grade (malignant) gliomas grow much more quickly. Grad IV gliomas are designated glioblastomas,

In several aspects the present invention further provides

1.1 A method for treating cancer in a subject in need thereof, comprising co-administering, concomitantly or in sequence, of a therapeutically effective amount of an mTOR inhibitor and a second drug substance which is a Raf kinase inhibitor.
1.2 A method for inhibiting tumor growth in a subject in need thereof comprising co-administering, concomitantly or in sequence, of a therapeutically effective amount of an mTOR inhibitor and a second drug substance which is a Raf kinase inhibitor.
1.3 A method for inducing tumor regression, e.g. (mass) reduction, in a subject in need thereof, comprising co-administering, concomitantly or in sequence, of a therapeutically effective amount of an mTOR inhibitor and a second drug substance which is a Raf kinase inhibitor.
1.4 A method for treating tumor invasiveness or symptoms associated with tumor growth in a subject in need thereof, comprising co-administering, concomitantly or in sequence, of a therapeutically effective amount of an mTOR inhibitor and a second drug substance which is a Raf kinase inhibitor.

In a series of further specific or alternative embodiments, the present invention also provides:

2.1 An mTOR inhibitor in combination with a Raf kinase inhibitor for use in any method as defined under 1.1 to 1.4 above.
3.1 An mTOR inhibitor in combination with a Raf kinase inhibitor for use in the preparation of a pharmaceutical composition for use in any method as defined under 1.1 to 1.4 above.
4.1 A pharmaceutical composition comprising an mTOR inhibitor in combination with a Raf kinase inhibitor together with one or more pharmaceutically acceptable diluents or carriers therefore, e.g. including fillers, binders, disintegrants, flow conditioners, lubricants, sugars or sweeteners, fragrances, preservatives, stabilizers, wetting agents and/or emulsifiers, solubilizers, salts for regulating osmotic pressure and/or buffers.
5.1 A pharmaceutical combination comprising a first drug substance which is an mTOR inhibitor, and a second drug substance which is a Raf kinase inhibitor.
5.2 A pharmaceutical combination comprising an amount of a first drug substance which is an mTOR inhibitor, and a second drug substance which is a Raf kinase inhibitor, to produce a synergistic therapeutic effect.

A pharmaceutical combination as used herein include fixed combinations, in which an mTOR inhibitor and a Raf kinase inhibitor are in the same formulation; kits, in which an mTOR inhibitor and a Raf kinase inhibitor in separate formulations are provided in the same package, e.g. with instruction for co-administration; and free combinations in which an mTOR inhibitor and a Raf kinase inhibitor are packaged separately, but instruction for concomitant or sequential administration are given.

One or more mTOR inhibitors and one or more RAF kinase inhibitors may be used in combination. Preferably, however, a combination of the present invention comprises one mTOR inhibitor and one Raf kinase inhibitor.

In further aspects, the present invention provides
6.1 A pharmaceutical package comprising a first drug substance which is an mTOR inhibitor and a second drug substance which is a Raf kinase inhibitor, beside instructions for combined administration.
6.2 A pharmaceutical package comprising an mTOR inhibitor beside instructions for combined administration with a Raf kinase inhibitor.
6.3 A pharmaceutical package comprising a Raf kinase inhibitor beside instructions for combined administration with an mTOR inhibitor.

Combinations according to the present invention are prone to provide synergistic effects.

In other aspects the present invention provides
7. A method for improving the therapeutic utility of a RAF kinase inhibitor, comprising co-administering, e.g. concomitantly or in sequence, of a therapeutically effective amount of a Raf kinase inhibitor and a second drug substance which is an mTOR inhibitor.
8. A method for improving the therapeutic utility of an mTOR inhibitor, comprising co-administering, e.g. concomitantly or in sequence, of a therapeutically effective amount of an mTOR inhibitor and a second drug substance which is a Raf kinase inhibitor.

Utility of a combination (composition) provided by the present invention, e.g. in any method provided by the present invention, may be determined by determining the ability of an mTOR inhibitor and of a Raf kinase inhibitor to affect the proliferation, migration, and invasion of human cancer cells. It can be shown that a combination of an mTOR inhibitor and a Raf kinase inhibitor blocks cell proliferation to a greater degree than either drug alone in cancer cell lines.

Similar results may also be obtained in in vivo animal studies.

Combination treatment according to the present invention may be further combined with other cancer treatment, e.g. beside a combined treatment with an mTOR inhibitor and a Raf kinase inhibitor, another drug substance which is prone to benefit the treatment may be administered. Such other drug includes cancer drugs.

In another aspect the present invention provides
A pharmaceutical combination, composition or pharmaceutical package according to the present further comprising an anticancer drug which anticancer drug is other than an mTOR inhibitor or a Raf kinase inhibitor.
Any method or use according to the present invention which comprises further using an anticancer drug which anticancer drug is other than an mTOR inhibitor or a Raf kinase inhibitor.

Anticancer drugs which are prone to be useful in combination therapy with a combination of the present invention e.g. include
i. a steroid; e.g. prednisone.
ii. an adenosine-kinase-inhibitor; which targets, decreases or inhibits nucleobase, nucleoside, nucleotide and nucleic acid metabolisms, such as 5-Iodotubercidin, which is also known as 7H-pyrrolo[2,3-d]pyrimidin-4-amine, 5-iodo-7-β-D-ribofuranosyl.
iii. an adjuvant; which enhances the 5-FU-TS bond as well as a compound which targets, decreases or inhibits, alkaline phosphatase, such as leucovorin, levamisole; and other adjuvants used in cancer chemotherapy adjuvants, such as mesna (Uromitexan®, Mesnex®).
iv. an adrenal cortex antagonist; which targets, decreases or inhibits the activity of the adrenal cortex and changes the peripheral metabolism of corticosteroids, resulting in a decrease in 17-hydroxycorticosteroids, such as mitotane.
v. an AKT pathway inhibitor; such as a compound which targets, decreases or inhibits Akt, also known as protein kinase B (PKB), such as deguelin, which is also known as 3H-bis[1]benzopyrano[3,4-b:6′,5′-e]pyran-7(7aH)-one, 13,13a-dihydro-9,10-dimethoxy-3,3-dimethyl-, (7aS, 13aS); and triciribine, which is also known as 1,4,5,6,8-pentaazaacenaphthylen-3-amine, 1,5-dihydro-5-methyl-1-β-6-D-ribofuranosyl; KP372-1 (QLT394).
vi. an alkylating agent; which causes alkylation of DNA and results in breaks in the DNA molecules as well as cross-linking of the twin strands, thus interfering with DNA replication and transcription of RNA, such as chlorambucil, chlormethine, cyclophosphamide, ifosfamide, melphalan, estramustine; nitrosureas, such as carmustine, fotemustine, lomustine, streptozocin (streptozotocin, STZ), BCNU; Gliadel; dacarbazine, mechlorethamine, e.g. in the form of a hydrochloride, procarbazine, e.g. in the form of a hydrochloride, thiotepa, temozolomide, nitrogen mustard, mitomycin, altretamine, busulfan, estramustine, uramustine. Cyclophosphamide can be administered, e.g., in the form as it is marketed, e.g., under the trademark CYCLOSTIN®; ifosfamide as HOLOXAN®, temozolomide as TEMODAR®, nitrogen mustard as MUSTARGEN®, estramustine as EMYCT®, streptozocin as ZANOSAR®.
vii. an angiogenesis inhibitor; which targets, decreases or inhibits the production of new blood vessels, e.g. which targets methionine aminopeptidase-2 (MetAP-2), macrophage inflammatory protein-1 (MIP-1alpha), CCL5, TGF-beta, lipoxygenase, cyclooxygenase, and topoisomerase, or which indirectly targets p21, p53, CDK2 and collagen synthesis, e.g. including fumagillin, which is known as 2,4,6,8-decatetraenedioic acid, mono[(3R,4S,5S,6R)-5-methoxy-4-[(2R,3R)-2-methyl-3-(3-methyl-2-butenyl)oxiranyl]-1-oxaspiro[2.5]oct-6-yl]ester, (2E,4E,6E,8E)-(9CI); shikonin, which is also known as 1,4-naphthalenedione, 5,8-dihydroxy-2-[(1R)-1-hydroxy-4-methyl-3-pentenyl]-(9CI); tranilast, which is also known as benzoic acid, 2-[[3-(3,4-dimethoxyphenyl)-1-oxo-2-propenyl]amino]; ursolic acid; suramin; benzamide or a derivative thereof, thalidomide, TNP-470.
viii. an anti-androgen; which blocks the action of androgens of adrenal and testicular origin which stimulate the growth of normal and malignant prostatic tissue, such as nilutamide; bicalutamide (CASODEX®), which can be formulated, e.g., as disclosed in U.S. Pat. No. 4,636,505.
ix. an anti-estrogen; which antagonizes the effect of estrogens at the estrogen receptor level, e.g. including an aromatase inhibitor, which inhibits the estrogen production, i.e. the conversion of the substrates adrostenedione and testosterone to estrone and estradiol, respectively,
   e.g. including atamestane, exemestane, formestane, aminoglutethimide, rogletimide, pyridoglutethimide, trilostane, testolactone, ketoconazole, vorozole, fadrozole, anastrozole, letrozole, toremifene; bicalutamide; flutamide; tamoxifen, tamoxifen citrate; tamoxifen; fulvestrant; raloxifene, raloxifene hydrochloride. Tamoxifen may be e.g. administered in the form as it is marketed, e.g., NOLVADEX®; and raloxifene hydrochloride is marketed as EVISTA®. Fulvestrant may be formulated as disclosed in U.S. Pat. No. 4,659,516 and is marketed as FASLODEX®.
x. an anti-hypercalcemia agent; which is used to treat hypercalcemia, such as gallium (III) nitrate hydrate; and pamidronate disodium.
xi. an antimetabolite; which inhibits or disrupts the synthesis of DNA resulting in cell death. Examples of an antimetabolite include, but are not limited to, DNA de-methylating agents and folic acid antagonists, e.g. methotrexate, pemetrexed, (permetrexed, Alimta®), raltitrexed; purins, e.g. 6-mercaptopurine, cladribine, clofarabine; fludarabine, thioguanine (thioguanine), 6-thioguanine, nelarabine (compound 506), tiazofurin (inhibits inosine monophosphate dehydrogenase and guanosine triphosphate pools), pentostatin (deoxycoformycin); cytarabine; flexuridine; fluorouracil; 5-fluorouracil (5-FU), floxuridine (5-FUdR), capecitabine; gemcitabine; gemcitabine hydrochloride; hydroxyurea (e.g. Hydrea®); DNA de-methylating agents, such as 5-azacytidine (Vidaza®) and decitabine; fluoroethylene deoxycytidine (FmdC), 5-aza-2′-deoxycytidine, troxacitabine (L-isomer cytosine analogue), edatrexate; Capecitabine and gemcitabine can be administered e.g. in the marketed form, such as XELODA® and GEMZAR®.
xii. an apoptosis inducer; which induces the normal series of events in a cell that leads to its death, e.g. selectively inducing the X-linked mammalian inhibitor of apoptosis protein XIAP, or e.g. downregulating BCL-xL; such as ethanol, 2-[[3-(2,3-dichlorophenoxy)propyl]amino]; gambogic acid; embelin, which is also known as 2,5-cyclohexadiene-1,4-dione, 2,5-dihydroxy-3-undecyl; arsenic trioxide arsenic trioxide (TRISENOX®).
xiii. an aurora kinase inhibitor; which targets, decreases or inhibits later stages of the cell cycle from the G2/M check point all the way through to the mitotic checkpoint and late mitosis; such as binucleine 2, which is also known as methanimidamide, N′-[1-(3-chloro-4-fluorophenyl)-4-cyano-1H-pyrazol-5-yl]-N,N-dimethyl.
xiv. a Bruton's Tyrosine Kinase (BTK) inhibitor; which targets, decreases or inhibits human and murine B cell development; such as terreic acid.
xv. a calcineurin inhibitor; which targets, decreases or inhibits the T cell activation pathway, such as cypermethrin, which is also known as cyclopropanecarboxylic acid, 3-(2,2-dichlorophenyl)-2,2-dimethyl-, cyano(3-phenoxyphenyl)methyl ester; deltamethrin, which is also known as cyclopropanecarboxylic aci, 3-(2,2-dibromoethenyl)-2,2-dimethyl-(S)cyano(3-phenoxyphenyl)methyl ester, (1R,3R); fenvalerate, which is also known as benzeneacetic acid, 4-chloro-α-(1-methylethyl) cyano(3-phenoxyphenyl)methyl ester; and Tyrphostin 8; but excluding cyclosporin or FK506.
xvi. a CaM kinase II inhibitor; which targets, decreases or inhibits CaM kinases; constituting a family of structurally related enzymes that include phosphorylase kinase, myosin light chain kinase, and CaM kinases I-IV; such as 5-isoquinolinesulfonic acid, 4-[(2S)-2-[(5-isoquinolinylsulfonyl)methylamino]-3-oxo-3-(4-phenyl-1-piperazinyl)propyl]phenyl ester (9CI); benzenesulfonamide, N-[2-[[[3-(4-chlorophenyl)-2-propenyl]methyl]amino]methyl]phenyl}-N-(2-hydroxyethyl)-4-methoxy.
xvii. a CD45 tyrosine phosphatase inhibitor, which targets, decreases or inhibits dephosphorylating regulatory pTyr residues on Src-family protein-tyrosine kinases, which aids in the treatment of a variety of inflammatory and immune disorders; such as phosphonic acid, [[2-(4-bromophenoxy)-5-nitrophenyl]hydroxymethyl].
xviii. a CDC25 phosphatase inhibitor; which targets, decreases or inhibits overexpressed dephosphorylate cyclin-dependent kinases in tumors; such as 1,4-naphthalenedione, 2,3-bis[(2-hydroyethyl)thio].
xix. a CHK kinase inhibitor; which targets, decreases or inhibits overexpression of the antiapoptotic protein Bcl-2; such as debromohymenialdisine. Targets of a CHK kinase inhibitor are CHK1 and/or CHK2. An example of a CHK kinase inhibitor includes, but is not limited to, debromohymenialdisine.
xx. a controlling agent for regulating genistein, olomucine and/or tyrphostins; such as daidzein, which is also known as 4H-1-benzopyran-4-one, 7-hydroxy-3-(4-hydroxyphenyl); Iso-Olomoucine, and Tyrphostin 1.
xxi. a cyclooxygenase inhibitor; e.g. including Cox-2 inhibitors; which targets, decreases or inhibits the enzyme Cox-2 (cyclooxygenase-2); such as 1H-indole-3-acetamide, 1-(4 chlorobenzoyl)-5-methoxy-2-methyl-N-(2-phenylethyl); 5-alkyl substituted 2-arylaminophenylacetic acid and derivatives, e.g. celecoxib (CELEBREX®), rofecoxib (VIOXX®), etoricoxib, valdecoxib; or a 5-alkyl-2-arylaminophenylacetic acid, e.g., 5-methyl-2-(2′-chloro-6′-fluoroanilino)phenyl acetic acid, lumiracoxib; and celecoxib.
xxiii. a cyclin dependent kinase inhibitor; which targets, decreases or inhibits cyclin dependent kinase playing a role in the regulation of the mammalian cell cycle; such as N9-isopropyl-olomoucine; olomoucine; purvalanol B, which is also known as Benzoic acid, 2-chloro-4-[[2-[[(1R)-1-(hydroxymethyl)-2-methylpropyl]amino]-9-(1-methylethyl)-9H-purin-6-yl]amino]-(9CI); roascovitine; indirubin, which is also known as 2H-indol-2-one, 3-(1,3-dihydro-3-oxo-2H-indol-2-ylidene)-1,3-dihydro-; kenpaullone, which is also known as indolo[3,2-d][1]benzazepin-6(5H)-one, 9-bromo-7,12-dihydro-; purvalanol A, which is also known as 1-Butanol, 2-[[6-[(3-chlorophenyl)amino]-9-(1-methylethyl)-9H-purin-2-yl]amino]-3-methyl-, (2R)—; indirubin-3′-monooxime. Cell cycle progression is regulated by a series of sequential events that include the activation and subsequent inactivation of cyclin dependent kinases (Cdks) and cyclins. Cdks are a group of serine/threonine kinases that form active heterodimeric complexes by binding to their regulatory subunits, cyclins. Examples of targets of a cyclin dependent kinase inhibitor include, but are not limited to, CDK, AHR, CDK1, CDK2, CDK5, CDK4/6, GSK3beta, and ERK.
xxiv. a cysteine protease inhibitor; which targets, decreases or inhibits cysteine protease which plays a vital role in mammalian cellular turnover and apotosis; such as 4-morpholinecarboxamide, N-[(1S)-3-fluoro-2-oxo-1-(2-phenylethyl)propyl]amino]-2-oxo-1-(phenylmethyl)ethyl].
xxv. a DNA intercalator; which binds to DNA and inhibits DNA, RNA, and protein synthesis; such as plicamycin, dactinomycin.
xxvi. a DNA strand breaker; which causes DNA strand scission and results in inhibition of DNA synthesis, inhibition of RNA and protein synthesis; such as bleomycin.
xxvii. an E3 Ligase inhibitor; which targets, decreases or inhibits the E3 ligase which inhibits the transfer of ubiquitin chains to proteins, marking them for degradation in the proteasome; such as N-((3,3,3-trifluoro-2-trifluoromethyl)propionyl)sulfanilamide.
xxviii. an endocrine hormone; which by acting mainly on the pituitary gland causes the suppression of hormones in males, the net effect being a reduction of testosterone to castration levels; in females, both ovarian estrogen and androgen synthesis being inhibited; such as leuprolide; megestrol, megestrol acetate.
xxix. compounds targeting, decreasing or inhibiting the activity of the epidermal growth factor family of receptor tyrosine kinases (EGFR, ErbB2, (HER-2), ErbB3, ErbB4 as homo- or heterodimers), such as compounds, proteins or antibodies which inhibit members of the EGF receptor tyrosine kinase family, e.g. EGF receptor, ErbB1, ErbB2, ErbB3 and ErbB4 or bind to EGF or EGF-related ligands, and are in particular those compounds, proteins or monoclonal antibodies generically and specifically disclosed in WO9702266, e.g. the compound of ex. 39, EP0564409, WO9903854, EP0520722, EP0566226, EP0787722, EP0837063, U.S. Pat. No. 5,747,498, WO9810767, WO9730034, WO9749688, WO9738983 and, especially, WO9630347, e.g. a compound known as CP 358774, WO9633980, e.g. a compound known as ZD 1839; and WO9503283, e.g. a compound known as ZM105180, Zemab®, e.g. including the dual acting tyrosine kinase inhibitor (ErbB1 and ErbB2) lapatinib (GSK572016), e.g. lapatinib ditosylate; AEE788, panituzumab, trastuzumab (HERCEPTIN®), cetuximab (Erbitux®), geftinib, OSI-774, CI-1033, EKB8569, GW-2016, E1.1, E2.4, E2.5, E6.2, E6.4, E2.11, E6.3 or E7.6.3, 7H-pyrrolo-[2,3-d]pyrimidine derivatives which are e.g. disclosed in WO03013541, erlotinib, vatanalib, gefitinib. Erlotinib can be administered in the form as it is marketed, e.g. TARCEVA®, and gefitinib as IRESSA®, human monoclonal antibodies against the epidermal growth factor receptor including ABX-EGFR.
xxx. an EGFR, PDGFR tyrosine kinase inhibitor; such as EGFR kinase inhibitors, e.g. zalutumumab, tyrphostin 23, tyrphostin 25, tyrphostin 47, tyrphostin 51 and tyrphostin AG 825; 2-propenamide, 2-cyano-3-(3,4-dihydroxyphenyl)-N-phenyl-(2E); tyrphostin Ag 1478; lavendustin A; 3-pyridineacetonitrile, α-[(3,5-dichlorophenyl)methylene]-, (αZ); an example of an EGFR, PDGFR tyrosine kinase inhibitor e.g. includes tyrphostin 46, ZK222584. PDGFR tyrosine kinase inhibitor including tyrphostin 46, SU101. Targets of an EGFR kinase inhibitor include guanylyl cyclase (GC-C) HER2, EGFR, PTK and tubulin.
xxxi. a farnesyltransferase inhibitor; which targets, decreases or inhibits the Ras protein; such as a-hydroxyfarnesylphosphonic acid; butanoic acid, 2-[[(2S)-2-[[(2S,3S)-2-[[(2R)-2-amino-3-mercaptopropyl]amino]-3-methylpentyl]oxy]-1-oxo-3-phenylpropyl]amino]4-(methylsulfonyl)-, 1-methylethyl ester, (2S); manumycin A; L-744,832 or DK8G557, tipifarnib (R115777), SCH66336 (lonafarnib), BMS-214662,
xxxii. a Flk-1 kinase inhibitor; which targets, decreases or inhibits Flk-1 tyrosine kinase activity; such as 2-propenamide, 2-cyano-3-[4-hydroxy-3,5-bis(1-methylethyl)phenyl]-N-(3-phenylpropyl)-(2E). A target of a Flk-1 kinase inhibitor includes, but is not limited to, KDR.
xxxiii. a Glycogen synthase kinase-3 (GSK3) inhibitor; which targets, decreases or inhibits glycogen synthase kinase-3 (GSK3); such as indirubin-3′-monooxime. Glycogen Synthase Kinase-3 (GSK-3; tau protein kinase I), a highly conserved, ubiquitously expressed serine/threonine protein kinase, is involved in the signal transduction cascades of multiple cellular processes. which is a protein kinase that has been shown to be involved in the regulation of a diverse array of cellular functions, including protein synthesis, cell proliferation, cell differentiation, microtubule assembly/disassembly, and apoptosis.
xxxiv. a histone deacetylase (HDAC) inhibitor; which inhibits the histone deacetylase and which possess anti-proliferative activity; such as compounds disclosed in WO0222577, especially N-hydroxy-3-[4-[[(2-hydroxyethyl)[2-(1H-indol-3-yl)ethyl]-amino]methyl]phenyl]-2E-2-propenamide, and N-hydroxy-3-[4-[[[2-(2-methyl-1H-indol-3-yl)-ethyl]-amino]methyl]phenyl]-2E-2-propenamide and pharmaceutically acceptable salts thereof; suberoylanilide hydroxamic acid (SAHA); [4-(2-amino-phenylcarbamoyl)-benzyl]-carbamic acid pyridine-3-ylmethyl ester and derivatives thereof; butyric acid, pyroxamide, trichostatin A, oxamflatin, apicidin, depsipeptide (FK228); depudecin; trapoxin, HC toxin, which a cyclic tetrapeptide (cyclo-[prolyl-alynyl-alanyl-2-amino-8-oxo-9,10-epoxydecanoyl]); sodium phenylbutyrate, suberoylanilide hydroxamic acid, suberoyl bis-hydroxamic acid; Trichostatin A, BMS-27275, pyroxamide, FR-901228, valproic acid, PXD101, Savicol®.
xxxv. a HSP90 inhibitor; which targets, decreases or inhibits the intrinsic ATPase activity of HSP90; degrades, targets, decreases or inhibits the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, e.g. a geldanamycin derivative; 17-allylamino-geldanamycin, 17-demethoxygeldanamycin (17AAG), other geldanamycin-related compounds; radicicol and HDAC inhibitors. Other examples of an HSP90 inhibitor include geldanamycin, 17-dimethoxy-17-(2-propenylamino). Potential indirect targets of an HSP90 inhibitor include FLT3, BCR-ABL, CHK1, CYP3A5*3 and/or NQ01*2. Nilotinib is an example of an BCR-ABL tyrosine kinase inhibitor.
xxxvi. a I-kappa B-alpha kinase inhibitor (IKK); which targets, decreases or inhibits NF-kappaB, such as 2-propanenitrile, 3-[(4-methylphenyl)sulfonyl]-(2E).
xxxvii. an insulin receptor tyrosine kinase inhibitor; which modulates the activities of phosphatidylinositol 3-kinase, microtubule-associated protein, and S6 kinases; such as hydroxyl-2-naphthalenylmethylphosphonic acid, LY294002.
xxxviii. a c-Jun N-terminal kinase (JNK) kinase inhibitor, which targets, decreases or inhibits Jun N-terminal kinase; such as pyrazoleanthrone and/or epigallocatechin gallate. Jun N-terminal kinase (JNK), a serine-directed protein kinase, is involved in the phosphorylation and activation of c-Jun and ATF2 and plays a significant role in metabolism, growth, cell differentiation, and apoptosis. A target for a JNK kinase inhibitor includes, but is not limited to, DNMT.
xxxix a microtubule binding agent; which acts by disrupting the microtubular network that is essential for mitotic and interphase cellular function; such as vinca alkaloids, e.g. vinblastine, vinblastine sulfate; vincristine, vincristine sulfate; vindesine; vinorelbine; taxanes, such as taxanes, e.g. docetaxel; paclitaxel; discodermolides; colchicine, epothilones and derivatives thereof, e.g. epothilone B or a derivative thereof. Paclitaxel is marketed as TAXOL®; docetaxel as TAXOTERE®; vinblastine sulfate as VINBLASTIN R.P®; and vincristine sulfate as FARMISTIN®. Also included are the generic forms of paclitaxel as well as various dosage forms of paclitaxel. Generic forms of paclitaxel include, but are not limited to, betaxolol hydrochloride. Various dosage forms of paclitaxel include, but are not limited to albumin nanoparticle paclitaxel marketed as ABRAXANE®; ONXOL®, CYTOTAX®. Discodermolide can be obtained, e.g., as disclosed in U.S. Pat. No. 5,010,099. Also included are Epotholine derivatives which are disclosed in U.S. Pat. No. 6,194,181, WO98/0121, WO9825929, WO9808849, WO9943653, WO9822461 and WO0031247. Especially preferred are Epotholine A and/or B.
xl. a mitogen-activated protein (MAP) kinase-inhibitor; which targets, decreases or inhibits Mitogen-activated protein, such as benzenesulfonamide, N-[2-[[[3-(4-chlorophenyl)-2-propenyl]methyl]amino]methyl]phenyl}-N-(2-hydroxyethyl)-4-methoxy. The mitogen-activated protein (MAP) kinases are a group of protein serine/threonine kinases that are activated in response to a variety of extracellular stimuli and mediate signal transduction from the cell surface to the nucleus. They regulate several physiological and pathological cellular phenomena, including inflammation, apoptotic cell death, oncogenic transformation, tumor cell invasion, and metastasis.
xli. a MDM2 inhibitor, which targets, decreases or inhibits the interaction of MDM2 and the p53 tumor suppressor; such as trans-4-iodo, 4′-boranyl-chalcone.
xlii. a MEK inhibitor; which targets, decreases or inhibits the kinase activity of MAP kinase MEK; such as sorafenib, e.g. Nexavar® (sorafenib tosylate), butanedinitrile, bis[amino[2-aminophenyl)thio]methylene]. A target of a MEK inhibitor includes, but is not limited to ERK. An indirect target of a MEK inhibitor includes, but is not limited to, cyclin D1.
xliii: a matrix metalloproteinase inhibitor (MMP) inhibitor; which targets, decreases or inhibits a class of protease enzyme that selectively catalyze the hydrolysis of polypeptide bonds including the enzymes MMP-2 and MMP-9 that are involved in promoting the loss of tissue structure around tumors and facilitating tumor growth, angiogenesis, and metastasis such as actinonin, which is also known as butanediamide, N-4-hydroxy-N1-[(1S)-1-[[(2S)-2-(hydroxymethyl)-1-pyrrolidinyl]carbonyl]-2-methylpropyl]-2-pentyl-, (2R)-(9CI); epigallocatechin gallate; collagen peptidomimetic and non-peptidomimetic inhibitors; tetracycline derivatives, e.g., hydroxamate peptidomimetic inhibitor batimastat; and its orally-bioavailable analogue marimastat, prinomastat, metastat, neovastat, tanomastat, TAA211, BMS-279251, BAY 12-9566, MMI270B or AAJ996. A target of a MMP inhibitor includes, but is not limited to, polypeptide deformylase.
xliv. a NGFR tyrosine-kinase-inhibitor; which targets, decreases or inhibits nerve growth factor dependent p140^{c-trk} tyrosine phosphorylation; such as tyrphostin AG 879. Targets of a NGFR tyrosine-kinase-inhibitor include, but are not limited to, HER2, FLK1, FAK, TrkA, and/or TrkC. An indirect target inhibits expression of RAF1.
xlv. a p38 MAP kinase inhibitor, including a SAPK2/p38 kinase inhibitor; which targets, decreases or inhibits p38-MAPK, which is a MAPK family member, such as phenol, 4-[4-(4-fluorophenyl)-5-(4-pyridinyl)-1H-imidazol-2-yl]. An example of a a SAPK2/p38 kinase inhibitor includes, but is not limited to, benzamide, 3-(dimethylamino)-N-[3-[(4-hydroxybenzoyl)amino]-4-methylphenyl]. A MAPK family member is a serine/threonine kinase activated by phosphorylation of tyrosine and threonine residues. This kinase is phosphorylated and activated by many cellular stresses and inflammatory stimuli, thought to be involved in the regulation of important cellular responses such as apoptosis and inflammatory reactions.
xlvi. a p56 tyrosine kinase inhibitor; which targets, decreases or inhibits p56 tyrosine kinase, which is an enzyme that is a lymphoid-specific src family tyrosine kinase critical for T-cell development and activation; such as damnacanthal, which is also known as 2-anthracenecarboxaldehyde, 9,10-dihydro-3-hydroxy-1 methoxy-9,10-dioxo, Tyrphostin 46. A target of a p56 tyrosine kinase inhibitor includes, but is not limited to, Lck. Lck is associated with the cytoplasmic domains of CD4, CD8 and the beta-chain of the IL-2 receptor, and is thought to be involved in the earliest steps of TCR-mediated T-cell activation.
xlvii. a PDGFR tyrosine kinase inhibitor; targeting, decreasing or inhibiting the activity of the C-kit receptor tyrosine kinases (part of the PDGFR family), such as targeting, decreasing or inhibiting the activity of the c-Kit receptor tyrosine kinase family, especially inhibiting the c-Kit receptor. Examples of targets of a PDGFR tyrosine kinase inhibitor includes, but are not limited to PDGFR, FLT3 and/or c-KIT; such as tyrphostin AG 1296; tyrphostin 9; 1,3-butadiene-1,1,3-tricarbonitrile,2-amino-4-(1H-indol-5-yl); N-phenyl-2-pyrimidine-amine derivative, e.g. imatinib, IRESSA®, MLN518. PDGF plays a central role in regulating cell proliferation, chemotaxis, and survival in normal cells as well as in various disease states such as cancer, atherosclerosis, and fibrotic disease. The PDGF family is composed of dimeric isoforms (PDGF-AA, PDGF-BB, PDGF-AB, PDGF-CC, and PDGF-DD), which exert their cellular effects by differentially binding to two receptor tyrosine kinases. PDGFR-α and PDGFR-β have molecular masses of −170 and 180 kDa, respectively.
xlviii. a phosphatidylinositol 3-kinase inhibitor; which targets, decreases or inhibits PI 3-kinase; such as wortmanin, which is also known as 3H-Furo[4,3,2-de]indeno[4,5-h]-2-benzopyran-3,6,9-trione, 11-(acetyloxy)-1,6b,7,8,9a,10,11,11b-octahydro-1-(methoxymethyl)-9a,11b-dimethyl-, (1S,6bR,9aS, 11R, 11 bR)-(9CI); 8-phenyl-2-(morpholin-4-yl)-chromen-4-one; quercetin, quercetin dihydrate. PI 3-kinase activity has been shown to increase in response to a number of hormonal and growth factor stimuli, including insulin, platelet-derived growth factor, insulin-like growth factor, epidermal growth factor, colony-stimulating factor, and hepatocyte growth factor, and has been implicated in processes related to cellular growth and transformation. An example of a target of a phosphatidylinositol 3-kinase inhibitor includes, but is not limited to, Pi3K.
xlix. a phosphatase inhibitor; which targets, decreases or inhibits phosphatase; such as cantharidic acid; cantharidin; and L-leucinamide, N-[4-(2-carboxylethenyl)benzoyl]glycyl-L-α-glutamyl-(E). Phosphatases remove the phosphoryl group and restore the protein to its original dephosphorylated state. Hence, the phosphorylation-dephosphorylation cycle can be regarded as a molecular “on-off” switch.
I. a platinum agent; which contains platinum and inhibit DNA synthesis by forming interstrand and intrastrand cross-linking of DNA molecules; such as carboplatin; cisplatin; oxaliplatin; cisplatinum; satraplatin and platinum agents such as ZD0473, BBR3464. Carboplatin can be administered, e.g., in the form as it is marketed, e.g. CARBOPLAT®; and oxaliplatin as ELOXATIN®.
Ii. a protein phosphatase inhibitor, including a PP1 and PP2 inhibitor and a tyrosine phosphatase inhibitor; which targets, decreases or inhibits protein phosphatase. Examples of a PP1 and PP2A inhibitor include cantharidic acid and/or cantharidin. Examples of a tyrosine phosphatase inhibitor include, but are not limited to, L-P-bromotetramisole oxalate; 2(5H)-furanone, 4-hydroxy-5-(hydroxymethyl)-3-(1-oxohexadecyl)-, (5R); and benzylphosphonic acid.

The term “a PP1 or PP2 inhibitor”, as used herein, relates to a compound which targets, decreases or inhibits Ser/Thr protein phosphatases. Type I phosphatases, which include PP1, can be inhibited by two heat-stable proteins known as Inhibitor-1 (I-1) and Inhibitor-2 (I-2). They preferentially dephosphorylate a subunit of phosphorylase kinase. Type II phosphatases are subdivided into spontaneously active (PP2A), CA²⁺-dependent (PP2B), and Mg²⁺-dependent (PP2C) classes of phosphatases. The term “tyrosine phosphatase inhibitor”, as used here, relates to a compounds which targets, decreases or inhibits tyrosine phosphatase. Protein tyrosine phosphatases (PTPs) are relatively recent additions to the phosphatase family. They remove phosphate groups from phosphorylated tyrosine residues of proteins. PTPs display diverse structural features and play important roles in the regulation of cell proliferation, differentiation, cell adhesion and motility, and cytoskeletal function. Examples of targets of a tyrosine phosphatase inhibitor include, but are not limited to, alkaline phosphatase (ALP), heparanase, PTPase, and/or prostatic acid phosphatase.
Iii. a PKC inhibitor and a PKC delta kinase inhibitor: The term “a PKC inhibitor”, as used herein, relates to a compound which targets, decreases or inhibits protein kinase C as well as its isozymes. Protein kinase C (PKC), a ubiquitous, phospholipid-dependent enzyme, is involved in signal transduction associated with cell proliferation, differentiation, and apoptosis. Examples of a target of a PKC inhibitor include, but are not limited to, MAPK and/or NF-kappaB. Examples of a PKC inhibitor include, but are not limited to, 1-H-pyrrolo-2,5-dione,3-[1-[3-(dimethylamino)propyl]-1H-indol-3-yl]4-(1H-indol-3-yl); bisindolylmaleimide IX; sphingosine, which is known as 4-octadecene-1,3-diol, 2-amino-, (2S,3R,4E)-(9CI); staurosporine, which is known as 9,13-Epoxy-1H,9H-diindolo[1,2,3-gh:3′,2′,1′-Im]pyrrolo[3,4-j][1,7]benzodiazonin-1-one, staurosporine derivatives such as disclosed in EP0296110, e.g. midostaurin; 2,3,10,11,12,13-hexahydro-10-methoxy-9-methyl-11-(methylamino)-, (9S,10R,11R,13R)-(9CI); tyrphostin 51; hypericin, which is also known as phenanthro[1,10,9,8-opqra]perylene-7,14-dione, 1,3,4,6,8,13-hexahydroxy-10,11-dimethyl-, enzastaurin (LY317615)stereoisomer, UCN-01, safingol, BAY 43-9006, bryostatin 1, perifosine; Ilmofosine; RO 318220 and RO 320432; GO 6976; Isis 3521; LY333531/LY379196. The term “a PKC delta kinase inhibitor”, as used herein, relates to a compound which targets, decreases or inhibits the delta isozymes of PKC. The delta isozyme is a conventional PKC isozymes and is Ca²⁺-dependent. An example of a PKC delta kinase inhibitor includes, but is not limited to, Rottlerin, which is also known as 2-Propen-1-one, 1-[6-[(3-acetyl-2,4,6-trihydroxy-5-methylphenyl)methyl]-5,7-dihydroxy-2,2-dimethyl-2H-1-benzopyran-8-yl]-3-phenyl-, (2E).
Iiii. a polyamine synthesis inhibitor; which targets, decreases or inhibits polyamines spermidine; such as DMFO, which is also known as (−)-2-difluoromethylornithine; N1, N12-diethylspermine 4 HCl. The polyamines spermidine and spermine are of vital importance for cell proliferation, although their precise mechanism of action is unclear. Tumor cells have an altered polyamine homeostasis reflected by increased activity of biosynthetic enzymes and elevated polyamine pools.
Iiv. a proteosome inhibitor; which targets, decreases or inhibits proteasome, such as aclacinomycin A; gliotoxin; PS-341; MLN 341; bortezomib; velcade. Examples of targets of a proteosome inhibitor include, but are not limited to, O(2)(−)-generating NADPH oxidase, NF-kappaB, and/or farnesyltransferase, geranyltransferase I.
Iv. a PTP1B inhibitor; which targets, decreases or inhibits PTP1B, a protein tyrosine kinase inhibitor; such as L-leucinamide, N-[4-(2-carboxylethenyl)benzoyl]glycyl-L-α-glutamyl-,(E).
Ivi. a protein tyrosine kinase inhibitor including a SRC family tyrosine kinase inhibitor; a Syk tyrosine kinase inhibitor; and a JAK-2 and/or JAK-3 tyrosine kinase inhibitor; The term “a protein tyrosine kinase inhibitor”, as used herein, relates to a compound which which targets, decreases or inhibits protein tyrosine kinases. Protein tyrosine kinases (PTKs) play a key role in the regulation of cell proliferation, differentiation, metabolism, migration, and survival. They are classified as receptor PTKs and non-receptor PTKs. Receptor PTKs contain a single polypeptide chain with a transmembrane segment. The extracellular end of this segment contains a high affinity ligand-binding domain, while the cytoplasmic end comprises the catalytic core and the regulatory sequences. Examples of targets of a tyrosine kinase inhibitor include, but are not limited to, ERK1, ERK2, Bruton's tyrosine kinase (Btk), JAK2, ERK ½, PDGFR, and/or FLT3. Examples of indirect targets include, but are not limited to, TNFalpha, NO, PGE2, IRAK, iNOS, ICAM-1, and/or E-selectin. Examples of a tyrosine kinase inhibitor include, but are not limited to, tyrphostin AG 126; tyrphostin Ag 1288; tyrphostin Ag 1295; geldanamycin; and genistein.
   Non-receptor tyrosine kinases include members of the Src, Tec, JAK, Fes, Abl, FAK, Csk, and Syk families. They are located in the cytoplasm as well as in the nucleus. They exhibit distinct kinase regulation, substrate phosphorylation, and function. Deregulation of these kinases has also been linked to several human diseases.

The term “a SRC family tyrosine kinase inhibitor”, as used herein, relates to a compound which which targets, decreases or inhibits SRC. Examples of a SRC family tyrosine kinase inhibitor include, but are not limited to, PP1, which is also known as 1H-pyrazolo[3,4-d]pyrimidin-4-amine, 1-(1,1-dimethylethyl)-3-(1-naphthalenyl); and PP2, which is also known as 1H-Pyrazolo[3,4-d]pyrimidin-4-amine, 3-(4-chlorophenyl)-1-(1,1-dimethylethyl).
The term “a Syk tyrosine kinase inhibitor”, as used herein, relates to a compound which targets, decreases or inhibits Syk. Examples of targets for a Syk tyrosine kinase inhibitor include, but are not limited to, Syk, STAT3, and/or STAT5. An example of a Syk tyrosine kinase inhibitor includes, but is not limited to, piceatannol, which is also known as 1,2-benzenediol, 4-[(1E)-2-(3,5-dihydroxyphenyl)ethenyl].
The term “a Janus (JAK-2 and/or JAK-3) tyrosine kinase inhibitor”, as used herein, relates to a compound which targets, decreases or inhibits janus tyrosine kinase. Janus tyrosine kinase inhibitor are shown anti-leukemic agents with anti-thrombotic, anti-allergic and immunosuppressive properties. Targets of a JAK-2 and/or JAK-3 tyrosine kinase inhibitor include, but are not limited to, JAK2, JAK3, STAT3. An indirect target of an JAK-2 and/or JAK-3 tyrosine kinase inhibitor includes, but is not limited to CDK2. Examples of a JAK-2 and/or JAK-3 tyrosine kinase inhibitor include, but are not limited to, Tyrphostin AG 490; and 2-naphthyl vinyl ketone.
Compounds which target, decrease or inhibit the activity of c-Abl family members and their gene fusion products, e.g. include PD180970; AG957; or NSC 680410.
Ivii. a retinoid; which target, decrease or inhibit retinoid dependent receptors; such as isotretinoin, tretinoin, alitretinoin, bexarotene, e.g. including an agent which interact with retinoic acid responsive elements on DNA, such as isotretinoin (13-cis-retinoic acid).
Iviii. a RNA polymerase II elongation inhibitor; which targets, decreases or inhibits insulin-stimulated nuclear and cytosolic p70S6 kinase in CHO cells; targets, decreases or inhibits RNA polymerase II transcription, which may be dependent on casein kinase II; and targets, decreases or inhibits germinal vesicle breakdown in bovine oocytes; such as 5,6-dichloro-1-beta-D-ribofuranosylbenzimidazole.
Ivix. a serine/threonine kinase inhibitor; which inhibits serine/threonine kinases; such as 2-aminopurine. An example of a target of a serine/threonine kinase inhibitor includes, but is not limited to, dsRNA-dependent protein kinase (PKR). Examples of indirect targets of a serine/threonine kinase inhibitor include, but are not limited to, MCP-1, NF-kappaB, elF2alpha, COX2, RANTES, IL8, CYP2A5, IGF-1, CYP2B1, CYP2B2, CYP2H1, ALAS-1, HIF-1, erythropoietin, and/or CYP1A1.
Ix. a sterol biosynthesis inhibitor; which inhibits the biosynthesis of sterols such as cholesterol; such as terbinadine. Examples of targets for a sterol biosynthesis inhibitor include, but are not limited to, squalene epoxidase, and CYP2D6. An example of a sterol biosynthesis inhibitor includes, but is not limited to, terbinadine.
Ixi. a topoisomerase inhibitor; including a topoisomerase I inhibitor and a topoisomerase II inhibitor. Examples of a topoisomerase I inhibitor include, but are not limited to, topotecan, gimatecan, irinotecan, camptothecin and its analogues, 9-nitrocamptothecin and the macromolecular camptothecin conjugate PNU-166148 (compound A1 in WO9917804); 10-hydroxycamptothecin e.g. the acetate salt; idarubicin, e.g. the hydrochloride; irinotecan, e.g. the hydrochloride; etoposide; teniposide; topotecan, topotecan hydrochloride; doxorubicin; epirubicin, epirubicin hydrochloride; 4′-epidoxorubicin, mitoxantrone, mitoxantrone, e.g. the hydrochloride; daunorubicin, daunorubicin hydrochloride, valrubicin, dasatinib (BMS-354825). Irinotecan can be administered, e.g., in the form as it is marketed, e.g., under the trademark CAMPTOSAR®. Topotecan can be administered, e.g., in the form as it is marketed, e.g., under the trademark HYCAMTIN®. The term “topoisomerase II inhibitor”, as used herein, includes, but is not limited to, the anthracyclines, such as doxorubicin, including liposomal formulation, e.g., CAELYX®, daunorubicin, including liposomal formulation, e.g., DAUNOSOME®, epirubicin, idarubicin and nemorubicin; the anthraquinones mitoxantrone and losoxantrone; and the podophillotoxines etoposide and teniposide. Etoposide is marketed as ETOPOPHOS®; teniposide as VM 26-BRISTOL®; doxorubicin as ADRIBLASTIN® or ADRIAMYCIN®; epirubicin as FARMORUBICIN® idarubicin as ZAVEDOS®; and mitoxantrone as NOVANTRON®.
Ixii. VEGFR tyrosine kinase inhibitor; which targets, decreases and/or inhibits the known angiogenic growth factors and cytokines implicated in the modulation of normal and pathological angiogenesis. The VEGF family (VEGF-A, VEGF-B, VEGF-C, VEGF-D) and their corresponding receptor tyrosine kinases [VEGFR-1 (Flt-1), VEGFR-2 (Flk-1, KDR), and VEGFR-3 (Flt-4)] play a paramount and indispensable role in regulating the multiple facets of the angiogenic and lymphangiogenic processes. An example of a VEGFR tyrosine kinase inhibitor includes 3-(4-dimethylaminobenzylidenyl)-2-indolinone. Compounds which target, decrease or inhibit the activity of VEGFR are especially compounds, proteins or antibodies which inhibit the VEGF receptor tyrosine kinase, inhibit a VEGF receptor or bind to VEGF, and are in particular those compounds, proteins or monoclonal antibodies generically and specifically disclosed in WO9835958, e.g. 1-(4-chloroanilino)-4-(4-pyridylmethyl) phthalazine or a pharmaceutical acceptable salt thereof, e.g. the succinate, or in WO0009495, WO0027820, WO0059509, WO9811223, WO0027819 and EP0769947; e.g. those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, Vol. 27, no. 1, pp 14-21, 1999; in WO0037502 and WO9410202; Angiostatin, described by M. S. O'Reilly et al, Cell 79, 1994, 315-328; Endostatin described by M. S. O'Reilly et al, Cell 88, 1997, 277-285; anthranilic acid amides; ZD4190; ZD6474 (vandetanib); SU5416; SU6668, AZD2171 (Recentin®); or anti-VEGF antibodies, such as anti-VEGF-alpha antibody tanibizumab (Lucentis®), or anti-VEGF receptor antibodies, e.g. RhuMab (bevacizumab, Avastin®). By antibody is meant intact monoclonal antibodies, polyclonal antibodies, multispecific antibodies formed from at least 2 intact antibodies, and antibodies fragments so long as they exhibit the desired biological activity. an example of an VEGF-R2 inhibitor e.g. includes axitinib,
Ixiii. a gonadorelin agonist, such as abarelix, goserelin, goserelin acetate,
Ixiv. a compound which induce cell differentiation processes, such as retinoic acid, alpha-, gamma- or 8-tocopherol or alpha-, gamma- or 8-tocotrienol.
Ixv. a bisphosphonate, e.g. including etridonic, clodronic, bludronic, pamidronic, alendronic, ibandronic, risedronic and zoledronic acid.
Ixvi. a heparanase inhibitor which prevents heparan sulphate degradation, e.g. PI-88,
Ixvii. a biological response modifier, preferably alymphokine or interferons, e.g. interferon alpha,
Ixviii. a telomerase inhibitor, e.g. telomestatin,
Ixix. mediators, such as inhibitors of catechol-O-methyltransferase, e.g. entacapone,
Ixx: inhibitors of Kinesin Spindle Protein (KSP), such as ispinesib,
Ixxi somatostabn or a somatostatin analogue, such as octreotide (Sandostatin® or Sandostatin LAR®).
Ixxii. Growth Hormone-Receptor Antagonists, such as pegvisomant, filgrastim or pegfilgrastim, or interferon alpha:
Ixxiii. monoclonal antibodies, e.g. useful for leukemia (AML) treatment, such as alemtuzumab (Campath®), rituximab/Rituxan®), gemtuzamab, (ozogamicin, Mylotarg®), epratuzumab.
Ixxiv. cytoxic antineoplastics, e.g. including altretamine, amsacrine, asparaginase (Elspar®), pegaspargase (PEG-L-asparaginase, Oncaspar®)), denileukin diftitox (Ontak®)), masoprocol,
Ixxv. a phosphodiesterase inhibitor, e.g. anagrelide (Agrylin®, Xagrid®).
Ixxvi. a cancer vaccine, such as MDX-1379.
Ixxvii. an immunosuppressive monoclonal antibody, e.g., monoclonal antibodies to leukocyte receptors or their ligands,
   e.g. CD20, such as rituximab (Rituxan®, ibritumomab tiuxetan conjugated to ¹¹¹In or ⁹⁰Y (Zevalin®), ¹³¹I tositumumab ( )Bexxar®), ofatumumab, ocrelizumab, hA20 (Immunomedics),
   CD22, such as epratuzumab, inotizumab ozogamicin (CMC544), CAT-3888,
   CD33, such as gemtuzumab (Mylotarg®,
   CD52, e.g. alemtuzumab (Campath-I®),
      CD11a, e.g. efalizumab (Raptiva®),
   CD3, e.g. visillzumab,
Ixxviii. antibodies against carcinoembryonic antigen (CEA), e.g. lapetuzumab, e.g. I apetuzumab-yttrium90, KSB-303, MFECP1, MFE-23,
Ixxix. mediators, e.g. inhibitors, of multiple receptor tyrosine kinases associated with tumour growth and angiogenesis, such as sunitinib (SU11248),
Ixxx. synthetic nonsteroidal estrogens, e.g. including diethylstilbestrol (DES, Stilboestrol®)),
Ixxxi. a recombinant binding molecule having at least a portion of the extracellular domain of CTLA4 or a mutant thereof, or an anti-CLA4 agent” e.g. including an at least extracellular portion of CTLA4 or a mutant thereof joined to a non-CTLA4 protein sequence, such as CTLA4Ig, (e.g. designated ATCC 68629) or a mutant thereof includes but is not limited to LEA29Y (belatacept); an anti-CTLA4 agent includes but is not limited to ipilimumab, ticilimumab.
Ixxxii. an alphaVbeta3 and alphaVbeta5 integrin receptor inhibitor, e.g. cilengitide (EMD121974)
Ixxxiii. A combination partner as indicated in WO2007030377 as a combination partner for a Raf kinase inhibitor.

Cancer treatment may be associated with radiotherapy. Cancer treatment may also be associated with vitamin or vitamin derivative (e.g. Leucovorin®) treatment.

1. A method for treating cancer in a subject in need thereof, comprising co-administering, concomitantly or in sequence, of a therapeutically effective amount of an mTOR inhibitor and a second drug substance which is a Raf kinase inhibitor.

2. A method for inhibiting growth of tumor in a subject in need thereof comprising co-administering, concomitantly or in sequence, of a therapeutically effective amount of an mTOR inhibitor and a second drug substance which is a Raf kinase inhibitor.

3. A method for inducing tumor regression in a subject in need thereof, comprising co-administering, concomitantly or in sequence, of a therapeutically effective amount of an mTOR inhibitor and a second drug substance which is a Raf kinase inhibitor.

4. A method for treating tumor invasiveness or symptoms associated with tumor growth in a subject in need thereof, comprising co-administering, concomitantly or in sequence, of a therapeutically effective amount of an mTOR inhibitor and a second drug substance which is a Raf kinase inhibitor.

5. (canceled)

6. (canceled)

7. A pharmaceutical composition comprising an mTOR inhibitor in combination with a Raf kinase inhibitor together with one or more pharmaceutically acceptable diluents or carriers therefore.

8. A pharmaceutical combination comprising a first drug substance which is an mTOR inhibitor, and a second drug substance which is a Raf kinase inhibitor.

9. A pharmaceutical combination comprising an amount of a first drug substance which is an mTOR inhibitor, and a second drug substance which is a Raf kinase inhibitor, to produce a synergistic therapeutic effect.

10. A pharmaceutical package comprising a first drug substance which is an mTOR inhibitor and a second drug substance which is a Raf kinase inhibitor, beside instructions for combined administration.

11. A pharmaceutical package comprising an mTOR inhibitor beside instructions for combined administration with a Raf kinase inhibitor.

12. A pharmaceutical package comprising a Raf kinase inhibitor beside instructions for combined administration with an mTOR inhibitor.

13. A method for improving the therapeutic utility of a Raf kinase inhibitor, comprising co-administering, e.g. concomitantly or in sequence, of a therapeutically effective amount of a RAF kinase inhibitor and a second drug substance which is an mTOR inhibitor.

14. A method for improving the therapeutic utility of an mTOR inhibitor, comprising co-administering, e.g. concomitantly or in sequence, of a therapeutically effective amount of an mTOR inhibitor and a second drug substance which is a Raf kinase inhibitor.
